(19) United States
(12) Patent Application Publication
(10) Pub. No.: US 2021/0301312 A1
(43) Pub. Date: Sep. 30, 2021

(54) SYNTHESIS OF DNA WITH IMPROVED YIELD

(71) Applicant: Touchlight IP Limited, London (GB)

(72) Inventors: Neil PORTER, London (GB); Paul James ROTHWELL, London (GB); Stefanie GEHRIG-GIANNINI, London (GB); Anna BABULA, London (GB); Thomas Antony James ADIE, London (GB)

(21) Appl. No.: 17/268,721

(22) PCT Filed: Aug. 16, 2019
(86) PCT No.: PCT/GB2019/052307

§ 371 Date: Feb. 16, 2021

(30) Foreign Application Priority Data
Aug. 17, 2018 (GB) 1813429.6

(51) Int. Cl. C12P 19/34

(52) U.S. Cl. CPC C12P 19/34

(57) ABSTRACT

The present invention relates to an improved process for synthesis of deoxyribonucleic acid (DNA), in particular cell-free enzymatic synthesis of DNA, preferably on a large scale, with an improved yield and/or with an improved efficiency. The species of cation present in the nucleotide salt as the counter-ion is critical to the yield, efficiency and fidelity of high yielding enzymatic DNA synthesis reaction. The processes herein use alternative cations as counter-ions for the ionic nucleotides, permitting the use of higher concentrations of nucleotides in DNA synthesis, and further allowing for more favourable reaction conditions to be used.

### FIELD

The present invention relates to an improved process for synthesis of deoxyribonucleic acid (DNA), in particular cell-free enzymatic synthesis of DNA, preferably on a large scale, with an improved yield and/or with an improved efficiency.

### BACKGROUND

Amplification of deoxyribonucleic acid (DNA) may be carried out through use of cell-based processes, such as by culture of bacteria propagating DNA to be amplified in fermenters. Cell-free enzymatic processes for amplification of DNA from a starting template have also been described, including the polymerase chain reaction and strand-displacement reactions.

In the past, amplification of DNA on a test scale has been performed using apparatus based on microtitre plates and robotically controlled pipettes to add reaction components as required. Such apparatus and processes are suitable for manufacturing small quantities of DNA for test purposes but do not provide sufficient quantities for other purposes. Large scale amplification and manufacture of specific nucleic acids and proteins has mostly been carried out through cell-based processes. Such methods are generally effective for production of very large volumes of product but costly to set up. Further, it is preferable to synthesise DNA in a cell-free environment for clinical and therapeutic purposes.

Large-scale DNA synthesis using chemical synthesis, such as phosphoramidite methods, are known, but are not without drawbacks. The reaction must generally be performed in organic solvents, many of which are toxic or otherwise hazardous. Another drawback to chemical synthesis is that it is not completely efficient, since following each nucleotide addition, some percentage of the growing oligonucleotide chains are capped, resulting in a yield loss. The total yield loss for the nucleotide chain being synthesised thus increases with each nucleotide added to the sequence. This inherent inefficiency in chemical synthesis of oligonucleotides ultimately limits the length of oligonucleotide that can be efficiently produced to oligonucleotides having 50 nucleic acid residues or less, and furthermore affects the accuracy of synthesis.

To date, biological catalysts such as polymerase have not been routinely exploited for industrial scale manufacture of DNA products in vitro and reactions have largely been limited to volumes at microliter scale. Scaling up processes using enzymatic synthesis of DNA has proved problematic, not least with the disappointing yield of DNA product.

The present applicants have previously addressed the ability of scaling-up using commercially available nucleotides. A new process was developed that involved adding fresh nucleotides to the reaction mixture as they became depleted or as the concentration of the product reached a threshold, as described in WO2016/034849, incorporated herein by reference. However, it was established that even higher yields could be achieved and the inventors have developed a new method described herein to further enhance the yield from enzymatic DNA synthesis.

Enzymatic DNA synthesis generally requires the use of a polymerase or polymerase-like enzyme to catalyse the addition of nucleotides to a nascent nucleic acid chain. Generally, a template DNA is required which is amplified in the reaction. However, it is also possible to perform template-free DNA synthesis, where the incorporation happens de novo.

It is important to note that due to the highly charged nature of nucleic acids, they are constantly surrounded by counter-ions to neutralise most of their charges to lessen the electrostatic repulsion between sections of sequence, so they can be condensed into neat, compact structures in the cell. The building blocks of nucleic acids, the nucleotides, are also an ionic species and require the presence of positive counter-ions in order to maintain electrical neutrality. Most, if not all, nucleotides are thus supplied as salts with a positive counter-ion. Since the nucleotide has four negative charges, salts are typically prepared with 2 divalent cations, or 4 monovalent cations. It will be apparent to those skilled in the art that as soon as a nucleotide salt is dispersed in water or other solvent, the salt may dissociate in solution into anionic and cationic components.

In general, nucleotides are supplied for DNA synthesis, amplification or sequencing as either a lithium or sodium salt. Lithium is generally preferred since these salts offer greater solubility and also stability to repeated freezing and thawing cycles than sodium salts and remain sterile due to the bacteriostatic activity of lithium towards various microorganisms, giving greater reliability and an extended shelf life. The use of these salts is so routine that those skilled in the art do not appear to question the counter-ion present with the nucleotide. Indeed, all of the nucleotides used in the Examples of WO2016/034849 are lithium salts of nucleotides, since these are marketed as the superior choice to those skilled in the art.

The present inventors have found that the species of cation present in the nucleotide salt as the counter-ion is however critical to the yield, efficiency and fidelity of high yielding enzymatic DNA synthesis reaction. This is rather unexpected, since commercially available nucleotides are available generally as only lithium or sodium salts. However, using alternative cations as counter-ions for the ionic nucleotides can have a great impact on the DNA synthesis reaction, as can be seen from the Examples included herein. Such an effect is surprising and unexpected, since it challenges the conventional understanding of the use of nucleotide salts and necessitates the design and production of new counter-ion salt dNTPs to perform the Examples.

### SUMMARY

The present invention relates to a process for cell-free production or synthesis of DNA. The process may allow for enhanced production of DNA compared to current methodologies, i.e. an increased or greater yield, a more efficient process or the ability to perform enzymatic DNA synthesis in an environment with fewer additional components than thought possible under current methodologies. This significantly increases productivity whilst reducing the cost of synthesising DNA, particularly on a large scale.

In general, the present invention relates to enzymatic DNA synthesis using a polymerase enzyme or other DNA synthesising enzyme, any of which can optionally be engineered to give it particular properties.

The present invention relates generally to isothermal methods of amplifying DNA that do not require temperature to be cycled via heating and cooling during amplification, but may allow for the use of heat to initially denature the DNA template. The invention preferably relates to the use of polymerase enzymes that are capable of replicating a DNA template via strand-displacement replication, independently or with the help of other enzymes.

The processes of the invention involve the use of nucleotides in the forms of salts. The salts include a positive counter-ion (cation). It is preferred that this counter-ion is a monovalent cation, i.e. it has a single positive charge due to the loss of one electron. In order to increase the yield and/or efficiency of the DNA synthesis, the monovalent cations may not be exclusively sodium or lithium ions or a mixture thereof, but at least a portion of the cations will have an ionic radius greater than that of the sodium ion. The presence of a proportion of sodium or lithium in the salts or in the process in general may be tolerated, but it is preferred that the salt comprises monovalent cations with an ionic radius greater than that of the sodium ion. It will be understood that as a nucleotide has four negative charges, in general four monovalent cations will be present in the salt to maintain electrical neutrality.

Accordingly there is provided a cell-free process for the enzymatic synthesis of DNA comprising the use of nucleotides supplied as salts, wherein said salts comprise a monovalent cation with an ionic radius that is greater than that of the sodium ion.

Accordingly there is provided a cell-free process for the enzymatic synthesis of DNA comprising the use of nucleotides in the form of salts, wherein said salts comprise monovalent cations with an ionic radius that is greater than that of the sodium ion.

It is preferred that the enzymatic DNA synthesis is for the manufacturing of DNA on a larger scale, i.e. for therapeutic or prophylactic use, rather than lab-scale amplification. It is in this scaling-up of laboratory scale amplification that the present inventors have found that it is not as simple as providing more substrate and other components and finding that the yield follows suit. For nucleotide salts with sodium and lithium, these were found to be inhibitory to the DNA synthesis enzymes at higher concentrations. The present inventors have found alternative ways around this inhibition. The present invention allows for the reaction set-up to be altered, and therefore for the use of nucleotide salts at a concentration equal to or greater than 10 mM. Thus, the process comprises the use of nucleotide salts at a concentration equal or greater than 10 mM, said concentration being determined when the nucleotides are added. The concentration is determined in the reaction mixture in which the process is performed. Thus, the concentration of the nucleotides is determined in the reaction mixture at the time that the nucleotides are added. The concentration is therefore the initial concentration or concentration at the initiation of the process.

Accordingly there is provided a cell-free process for the enzymatic DNA synthesis comprising the use of nucleotides supplied as salts at a concentration of at least 10 mM, wherein said salts comprise a monovalent cation with an ionic radius that is greater than that of the sodium ion.

Accordingly there is provided a cell-free process for the enzymatic DNA synthesis comprising the use of nucleotides in the form of salts at a concentration of at least 10 mM, wherein said salts comprise a monovalent cation with an ionic radius that is greater than that of the sodium ion.

Any nucleotide salt as described here may have up to four monovalent cations present to maintain electrical neutrality.

Further, it is possible to use mixtures of nucleotide salts in the enzymatic DNA synthesis or cell-free process.

Accordingly there is provided a cell-free process for the enzymatic DNA synthesis comprising the use of nucleotides supplied as salts, wherein said nucleotides are either:

(a) in the form of a salt with a single monovalent cation whose ionic radius is greater than that of the sodium ion, or

(b) in the form of salts with 2 or more different monovalent cations, wherein at least one of the cations has an ionic radius greater than that of the sodium ion.

The nucleotides in this aspect may be provided at a concentration of greater than 10 mM.

Accordingly there is provided a cell-free process for the enzymatic DNA synthesis comprising the use of nucleotides in the form of salts, wherein the nucleotides are either:

(a) in the form of a salt with a single monovalent cation whose ionic radius is greater than that of the sodium ion, or

(b) in the form of salts with 2 or more different monovalent cations, wherein at least one of the cations has an ionic radius greater than that of the sodium ion.

The nucleotides in this aspect are present at concentration of greater than 10 mM.

As used herein, “single monovalent cation” means a single monovalent cation species, of which there may be up to four used to counter-balance the negative charge on the nucleotide ion.

Alternatively written there is provided:

A cell-free process for the enzymatic synthesis of DNA comprising the use of nucleotides in the form of salts, wherein said salts are present at a concentration of at least 10 mM and are either:

(a) in the form of a salt comprising a monovalent cation whose ionic radius is greater than that of the sodium ion, or

(b) in the form of two or more salts, each salt comprising different monovalent cations, wherein at least one of the cations has an ionic radius greater than that of the sodium ion.

The enzymatic DNA synthesis may involve any enzyme capable of synthesising DNA, including a polymerase or a modified polymerase. The polymerase may be from any of the known families of DNA polymerase, including families A, B, C, D, X, Y and RT. An example of a DNA polymerase from family X is terminal deoxynucleotidyl transferase.

The enzymatic DNA synthesis may occur de novo without the use of a template.

The enzymatic DNA synthesis may involve a template, such as a DNA template.

The enzymatic DNA synthesis may take place in a reaction mixture, comprising the components described here.

Alternatively written, there is provided a cell-free process for synthesising DNA comprising contacting a DNA template with at least one polymerase in the presence of one or more nucleotides in the form of a salt to form a reaction mixture, wherein said nucleotides are present at a concentration of at least 10 mM and are either:

(a) in the form of a salt with a single monovalent cation whose ionic radius is greater than that of the sodium ion, or

(b) in the form of salts with 2 or more different monovalent cations, wherein at least one of the cations has an ionic radius greater than that of the sodium ion.

Alternatively put, the nucleotide salts include monovalent cations that are not exclusively sodium or lithium, but a significant proportion are nucleotide salts which include cations with an ionic radius greater than that of the sodium ion. Accordingly there is provided a cell-free process for synthesising DNA comprising contacting a DNA template with at least one polymerase in the presence of one or more nucleotides in the form of a salt with a monovalent cation to form a reaction mixture, wherein said nucleotides are present at a concentration of at least 10 mM and are not exclusively sodium or lithium.

It is preferred that when the concentration of the nucleotide or nucleotide salt is referred to, this is the concentration of nucleotide (or salt thereof) when the process begins, i.e. the starting or initial concentration of nucleotide (or nucleotide salt). Thus, it is the concentration after addition to the reaction mixture. It will be appreciated that addition of other components can be made during the process; such additions may dilute the concentration of the nucleotides/nucleotide salts, unless further nucleotides are supplied to replenish the concentration. Further, since the nucleotide/nucleotide salts will be used or consumed by the process, i.e. the DNA synthesis reaction, the concentration of the nucleotide/nucleotide salts will fall as the process progresses. In certain embodiments, further nucleotides/nucleotide salts may be added as the process progresses in order to replenish substrates for the enzymatic reaction.

The inventors have surprisingly found that if the nucleotide salts include monovalent cations with an ionic radius greater than that of the sodium ion, the requirement for a divalent cation in the synthesis is reduced. Convention dictates, for example, that magnesium (a divalent cation) is present in DNA synthesis reactions at a minimum ratio of at least 1:1 with the nucleotide salts. This is because magnesium is required at the active site of some polymerase enzymes; it may form a complex with the nucleotide prior to incorporation and further may form its own salt with the phosphate ionic species released during DNA synthesis. However, under certain conditions, the present inventors have developed a method wherein the requirement for magnesium or other divalent cations is much reduced. This is important, since reducing the components included in the DNA synthesis notably reduces costs, but furthermore a higher concentration of magnesium is related to a decreased fidelity in DNA synthesis.

The divalent cations may comprise one or more metals selected from the list consisting of: Mg²⁺, Be²⁺, Ca²⁺, Sr²⁺, Mn²⁺ or Zn²⁺, preferably Mg²⁺ or Mn²⁺. The ratio between the metal cations and the nucleotide salts may be about 1:1 in the reaction mixture. Ratios lower than 1:1 are desirable and are preferable in DNA synthesis since ratios higher than 1:1 can lead to some infidelity in DNA synthesis. The divalent cations may be provided to the enzymatic DNA synthesis in the form of any suitable salt.

The invention therefore also relates to an enzymatic DNA synthesis which is performed under conditions of reduced divalent cations comprising the use of nucleotide salts with a monovalent cation or cations with an ionic radius greater than that of the sodium ion. Reduced in this context is in comparison to an identical reaction where lithium or sodium ions are present in the nucleotide salt.

The inventors have surprisingly found that the use of nucleotide salts with alternative counter-ions such as ammonium and caesium ions in the processes of the invention reduces the requirement for buffering agents to be included in the enzymatic DNA synthesis. This again is advantageous, since it reduces the cost of the synthesis reaction and could be beneficial for DNA synthesis for therapeutic use.

Further, the method developed here by the present inventors can be performed in a large range of conditions with respect to the other components present. These conditions range from a conventional level of buffering to the provision of no further buffering agents, effectively performing the reaction with the required components in water. The required components may include the DNA synthesising enzyme, i.e. a polymerase, the nucleotide salts, and a divalent cation (as a salt), with optional additional components required depending on the conditions of the reaction, selected from a template, a denaturing agent, a pyrophosphatase, or one or more primers. These components may form the reaction mixture.

Thus, the provision to the process, i.e. the reaction mixture, of at least a proportion of nucleotides as salts with a monovalent positive counter-ion (cation) with an ionic radius greater than that of the sodium ion is advantageous, since this surprisingly allows for improved DNA yield and/or an improved efficiency of conversion of the nucleotides into DNA. These improvements may be compared to an analogous reaction mixture where all the nucleotides are supplied as conventional salts alone, for example as solely lithium or sodium salts, or a mixture of these two ions. The provision of different nucleotide salts to those conventionally used has some further surprising advantages, such as the ability to lower the concentration of buffering agents in the reaction mixture, in some instances to zero, and to lower the requirement for a divalent cation co-factor for the reaction mixture, most notably magnesium.

In one aspect, a template directs the enzymatic DNA synthesis in the processes. This template may be a DNA template. The amplification of the template is preferably via strand-displacement. The amplification of the template is preferably isothermal, i.e. there is no requirement to cycle between low and high temperatures to progress the amplification. In this scenario, heat may be used at the start to denature the template, if required, or the template may be denatured by chemical means. However, once the template has been denatured, if appropriate, to allow any primers to enter between double stranded template, the temperature may be maintained at a range of temperatures that do not affect the denaturation of the template and product. Isothermal temperature conditions require that the reaction is not heated to a point where the template and products denature (compared to PCR which requires cycles of heat to denature the template and product). Generally such reactions are performed at a constant temperature, depending on the preference of the enzyme itself. The temperature may be any suitable temperature for the enzyme.

The cell-free process preferably involves amplification of the template via strand displacement replication. This synthesis releases a single stranded DNA, which may in turn be copied into double stranded-DNA, using the polymerase. The term strand displacement describes the ability to displace downstream DNA encountered during synthesis, wherein the polymerase opens the double-stranded DNA in order to extend the nascent single strand. DNA polymerases with varying degrees of strand displacement activity are available commercially. Alternatively, strand displacement can be achieved by supplying a DNA polymerase and a separate helicase. Replicative helicases may open the duplex DNA and facilitate the advancement of the leading-strand polymerase.

Independently, optional features of any aspect of the invention may be: The template may be circular. The strand displacement amplification of said DNA template may be carried out by rolling circle amplification (RCA). The polymerase may be Phi29, or a variant thereof. The amplification of DNA may be isothermal amplification, i.e. at a constant temperature. The one or more primers may be random primers. A pair or set of primers may be used. The synthesised DNA may comprise concatamers comprising tandem units of DNA sequence amplified from the DNA template. The DNA template may be a closed linear DNA; preferably the DNA template is incubated under denaturing conditions to form a closed circular single stranded DNA.

The quantity of DNA that may be synthesised is equal to or higher than 3 g per litre of reaction mixture, notably 16 g/l or more, preferably up to 30 g/l and beyond.

The amount of DNA that may be synthesised may exceed 60% of the calculated maximum yield for the reaction mixture. Preferably, the amount of DNA that may be synthesised may exceed 80% of the calculated maximum yield. The calculated maximum yield is based upon the theoretical yield should all nucleotides be incorporated into a product, and this can be calculated by those skilled in the art.

The efficiency of DNA synthesis from the nucleotides (or nucleotide salts) may be described as the percentage of nucleotides or salts thereof supplied to the reaction mixture which are successfully incorporated into a product over the course of the reaction.

The cell-free process requires at least one nucleotide. One or more further nucleotides may then be added. The nucleotides or further nucleotides are deoxyribonucleoside triphosphates (dNTPs), or a derivative or modified version thereof. The nucleotides or further nucleotides are one or more of deoxyadenosine triphosphate (dATP), deoxyguanosine triphosphate (dGTP), deoxycytidine triphosphate (dCTP), deoxythymidine triphosphate (dTTP) and derivatives thereof. The nucleotides or further nucleotides are provided as salts thereof. Each individual nucleotide salt may comprise up to four monovalent cations to maintain electrical neutrality. The nucleotide salts used in the process may include one or more monovalent cations, i.e. one or more species of monovalent cation, and it is preferred that most if not all of said monovalent cations have an ionic radius which is greater than that of the sodium ion. It will be appreciated that these may dissociate in solution, and therefore contribute to the presence of the cation in the process.

It is preferred that the concentration of nucleotides or salts thereof in the process, i.e. in the reaction mixture, may be more than 10 mM and up to at least 100 mM. Such concentrations are important in the production of higher yields of DNA, which in the case of the two concentrations given can be as high as 3 g/l to 30 g/l. It is preferred that the concentration of nucleotide or salts thereof stated is at the start of the process, i.e. is the starting or initial concentration of nucleotides or salts thereof in the reaction mixture, which also includes the enzyme necessary for DNA synthesis. Subsequent addition of further components may reduce this concentration, and their use by the DNA synthesis enzyme will also reduce the concentration from the starting concentration. Those skilled in the art will be aware of how to calculate the concentration of nucleotides/nucleotide salts as the process is prepared, based upon the volume of the other components and the stock nucleotide salt solution/powder used.

The terms nucleotide and nucleotide salt are used interchangeably in the art, since all nucleotides are inherently supplied as salts.

The process may be a batch process or a continuous flow process. The batch may be a closed batch (i.e. all of the reaction components are provided at the start of the DNA synthesis) or further components can be supplied to the reaction as required during the process, such as described in WO2016/034849, incorporated herein by reference. Should further additions be required, this will dilute the concentration of the nucleotide or nucleotide salts, unless further nucleotide salts are added to replenish the concentration.

The present inventors have found that each of the different counter-ions may add a particular characteristic to the enzymatic DNA synthesis reaction. For example, the use of nucleotide salts with caesium ions result in enzymatic DNA synthesis in the presence of reduced levels of magnesium. Further, the use of ammonium ions in nucleotide salts has resulted in the use of some high concentrations of nucleotides, with the Examples showing DNA synthesis at 80 mM nucleotide concentration.

The inventors are not aware of the use of several of these cations in nucleotide salts previously, underpinned by the lack of their ready availability from a commercial source. These nucleotide salts can be custom ordered from nucleotide manufacturers if required.

Accordingly, the use of nucleotide salts comprising any one of caesium, ammonium, ammonium derivatives or rubidium cations in the enzymatic cell-free synthesis of DNA forms part of the invention. Thus, use of nucleotide salts comprising these ions forms part of the invention.

The enzymatic cell-free synthesis of DNA with such ions can be carried out in the presence of low levels of divalent cation, less than about 1:1, preferably at a ratio of 0.2:1 to 0.8:1 divalent cation to nucleotide, preferably 0.2:1 to 0.5:1. The ions are the counter-ion in a nucleotide salt.

The enzymatic cell-free synthesis of DNA with such ions can be carried out in minimal buffering agents, in which no additional salts which have been shown to enhance DNA synthesis or assist in primer binding, or detergents, are added. This minimal buffer may comprise an agent (a buffering agent) to stabilise the pH. The minimal buffering agent may contain a very small amount of cations provided by the presence of a chemical used to denature the template, such as sodium, potassium or ammonium hydroxide. The ions are the counter-ion in a nucleotide salt.

Should a low level of magnesium ions be desirable in the enzymatic DNA synthesis, the inventors have found that a reliable nucleotide for such a synthesis is a nucleotide salt with caesium ions.

Accordingly, the present invention provides an enzymatic cell-free synthesis of DNA wherein maintaining a ratio of divalent ions to nucleotides of 0.5:1 or below is required, comprising the use of nucleotide salts comprising caesium ions.

Further advantages are described below.

### BRIEF DESCRIPTION OF THE DRAWINGS

The present invention will be described further below with reference to exemplary embodiments and the accompanying drawings, in which:

FIG. 1A to 1E are plots showing results obtained with experiments using varied starting concentrations of nucleotide salts with different counter-ions and different initial/starting concentrations of magnesium ions (as MgCl₂) in a DNA synthesis reaction. Each plot shows the obtained raw DNA yield (g/l) versus the theoretical DNA yield (g/l) corresponding to the total initial/starting nucleotide salt concentration (mM). On all of the plots the dashed line shows 80% efficiency of conversion of the nucleotide salts into DNA, the solid line shows 100% efficiency of conversion. FIG. 1A depicts the DNA synthesis results obtained using lithium-dNTPs; FIG. 1B the results achieved with sodium-dNTPs, FIG. 1C the results obtained with potassium-dNTPs FIG. 1D the results achieved with ammonium-dNTPs, and FIG. 1E, the results when using caesium-dNTPs;

FIG. 2 is a graph showing data from DNA synthesis experiments and is a plot of dNTP salt concentration (mM) that gave the maximum raw DNA yield against different reaction concentrations of magnesium ions. Shown are the results for nucleotides salts using: lithium, sodium, potassium, ammonium and caesium as the counter-ions. The graph is divided into three sections, highlighting the results where the ratio of magnesium ion to nucleotide salt is less than 0.5, a section where the ratio is 0.5 and 1 and a final section where the ratio exceeds 1. Also shown are the thresholds for these sections—a dotted line indicating 0.5:1 ratio and a solid line indicating 1:1 ratio of magnesium ion to nucleotide (dNTP);

FIG. 2 is a graph showing data obtained from DNA synthesis experiments. In the examples, the DNA yield was measured for various DNA synthesis reactions conducted with a fixed starting concentration of nucleotide salts using various counter-ions and increasing concentrations of magnesium chloride. The graph shows raw DNA yield (g/1) plotted against magnesium chloride concentration for all of the tested nucleotide salts;

FIG. 3 is a graph showing data from DNA synthesis reactions using rolling circle amplification performed in minimal buffer using varying starting concentrations of nucleotide salts. Plotted is the raw DNA yield (g/1) versus initial/starting dNTP salt concentrations (mM);

FIG. 4 is a plot from DNA synthesis experiments using rolling circle amplification of a DNA template, during which it was tested whether the presence of additional monovalent cations in the reaction mixture would have an effect on the DNA synthesis reaction. In this experiment, ammonium-nucleotide salts were used, and monovalent cation chloride salts as shown were also included in the reaction mixture. The starting ratio of ammonium dNTP salts to monovalent chloride salt was 1:4; since one monovalent cation was provided for each ammonium ion present on the dNTP (of which there are four). Thus, the starting ratio of ammonium ion (on the dNTP salt) to monovalent cation is 1:1. The starting concentration of magnesium is also varied, 5 mM, 10 mM, 20 mM and 40 mM corresponding respectively to the 17.5 mM, 25 mM, 35 mM and 50 mM concentrations of ammonium counter-ion dNTPs shown in the Figure. Plotted is the raw DNA yield for the indicated concentrations of ammonium counter-ion dNTPs (NH₄-dNTPs) in the presence of counter-ion chloride salts of monovalent cations including lithium, sodium, potassium, ammonium and caesium, the control being no additional salt.

FIGS. 5a and 6b are the results of several pH determination experiments comparing the pH of a reaction mixture lacking polymerase, template and primer at various starting concentrations of nucleotide salts. Variable initial magnesium chloride (MgCl₂) concentrations are used. The plots show the measured pH against the concentration of the nucleotide salt at the stated initial magnesium chloride concentration. No DNA synthesis has occurred. FIG. 6a shows a plot of the data for caesium-dNTPs and FIG. 6b shows a plot of the data for ammonium-dNTPs; and

FIG. 7 is a plasmid map proTLx-K B5X4 LUX ST(AT) as used in the Examples. Shown are processing sites (TeIRL), Luc 2 reporter gene, Kanamycin resistance gene, CMV promoter and pUC ori.

### DETAILED DESCRIPTION

The present invention relates to cell-free processes for large scale synthesis of DNA. The processes of the invention may allow for a high throughput synthesis of DNA.

The deoxyribonucleic acid (DNA) synthesised according to the present invention can be any DNA molecule. The DNA may be single stranded or double stranded. The DNA may be linear. The DNA may be processed to form circles, particularly minicircles, single stranded closed circles, double stranded closed circles, double stranded open circles, or closed linear double stranded DNA. The DNA may be allowed to form, or processed to form a particular secondary structure, such as, but not limited to hairpin loops (stem loops), imperfect hairpin loops, pseudoknots, or any one of the various types of double helix (A-DNA, B-DNA, or Z-DNA). The DNA may also form hairpins and aptamer structures.

The DNA synthesised may be of any suitable length. Lengths of up to or exceeding 77 kilobases may be possible using the processes of the invention. More particularly, the length of DNA which may be synthesised according to the processes of the invention may be in the order of up to 60 kilobases, or up to 50 kilobases, or up to 40 kilobases, or up to 30 kilobases. Preferably the DNA synthesised may be 100 bases to over 77 kilobases, 500 bases to 60 kilobases, 200 bases to 20 kilobases, more preferably 200 bases to 15 kilobases, most preferably 2 kilobases to 15 kilobases.

The amount of DNA synthesised according to the processes of the present invention may exceed 3 g/l. It is preferred that the amount of DNA synthesised is greater than 3, 4, 5, 6, 7, 8, 9, 10, 11, 12, 13, 14, 15, 16, 17, 18, 19, 20, 21, 22, 23, 24, 25, 26, 27, 28, 29 or 30 g/l or more. A preferred amount of DNA synthesised is 5 g/l. The amount of DNA produced may be described as industrial or commercial quantities, on a large-scale or mass production. The DNA produced by the processes of the invention may be uniform in quality, namely in DNA length and sequence. The processes may thus be suitable for large scale synthesis of DNA. The process may be uniform in terms of fidelity of synthesis.

Alternatively, the amount of DNA produced in the synthesis reaction may be compared to the theoretical maximum yield which would be achieved if 100% nucleotides were incorporated into the synthesised DNA. The methods of the invention not only improve the total yield obtained, but also the efficiency of the process, meaning that more of the supplied nucleotides are incorporated into the synthesised DNA product than in previous methods. Yields obtainable by the methods of the invention exceed 50% of the theoretical maximum, up to and exceeding 90% of the theoretical maximum. Therefore, the proportion of the theoretical maximum yield achieved by methods of the invention include 50%, 55%, 60%, 65%, 70%, 75%, 80%, 85%, 90% and 95% or greater. Conventionally, using commercially available nucleotide salts, yields achieved could be disappointing, due to effects of the ions that may be inhibitory to the process.

The DNA is synthesised in an enzymatic reaction. This enzymatic synthesis may involve the use of any DNA synthesising enzyme, most notably a polymerase enzyme or a modified polymerase enzyme. These are discussed further below. The DNA synthesis may be de novo and not require a template. The enzymatic synthesis may also require the use of a template for the DNA synthesis. This template can be any suitable nucleic acid depending on the polymerase, but is preferably a DNA template.

The template may be any suitable template, merely providing the instructions for the synthesis of the DNA by including a particular sequence. The template may be single stranded (ss) or double stranded (ds). The template may be linear or circular. The template may include natural, artificial or modified bases or a mixture thereof.

The template may comprise any sequence, either naturally derived or artificial.

The template may be of any suitable length. Particularly, the template may be up to 60 kilobases, or up to 50 kilobases, or up to 40 kilobases, or up to 30 kilobases. Preferably the DNA template may be 10 bases to 100 bases, 100 bases to 60 kilobases, 200 bases to 20 kilobases, more preferably 200 bases to 15 kilobases, most preferably 2 kilobases to 15 kilobases.

The template may be provided in an amount sufficient for use in the process by any method known in the art. For example, the template may be produced by PCR.

The whole or a selected portion of the template may be amplified in the process.

The template may comprise a sequence for expression. The DNA may be for expression in a cell (i.e. a transfected cell in vitro or in vivo), or may be for expression in a cell free system (i.e. protein synthesis). The sequence for expression may be for therapeutic purposes, i.e. gene therapy of a DNA vaccine. The sequence for expression may be a gene, and said gene may encode a DNA vaccine, a therapeutic protein and the like. The sequence may comprise a sequence which is transcribed into an active RNA form, i.e. a small interfering RNA molecule (siRNA).

If required, the template may be contacted with at least one polymerase, as described below.

The enzymatic DNA synthesis reaction may require at least one DNA synthesis enzyme. Preferably, the enzyme is a polymerase. Polymerases link together nucleotides to form a DNA polymer. One, two, three, four or five different enzymes and/or polymerases may be used. The polymerase may be any suitable polymerase from any family of polymerases, such that it synthesises polymers of DNA. The polymerase may be a DNA polymerase. Any DNA polymerase may be used, including any commercially available DNA polymerase. Two, three, four, five or more different DNA polymerases may be used, for example one which provides a proofreading function and one or more others which do not. DNA polymerases having different mechanisms may be used e.g. strand displacement type polymerases and DNA polymerases replicating DNA by other methods. A suitable example of a DNA polymerase that does not have strand displacement activity is T4 DNA polymerase. Template-independent polymerases may be used, such as terminal transferases.

Modified polymerases may also be used. These may have been engineered to modify their characteristics, such as to remove their dependency upon a template, to change their temperature dependency or to stabilise the enzyme for use in vitro.

A polymerase may be highly stable, such that its activity is not substantially reduced by prolonged incubation under process conditions. Therefore, the enzyme preferably has a long half-life under a range of process conditions including but not limited to temperature and pH. It is also preferred that a polymerase has one or more characteristics suitable for a manufacturing process. The polymerase preferably has high fidelity, for example through having proofreading activity. Furthermore, it is preferred that a polymerase displays high processivity, high strand-displacement activity and a low Kₘ for dNTPs and DNA. A polymerase may be capable of using circular and/or linear DNA as template. The polymerase may be capable of using dsDNA or ssDNA as a template. It is preferred that a polymerase does not display DNA exonuclease activity that is not related to its proofreading activity.

The skilled person can determine whether or not a given polymerase displays characteristics as defined above by comparison with the properties displayed by commercially available polymerases, e.g. Phi29 (New England Biolabs, Inc., Ipswich, Mass., US), Deep Vent® (New England Biolabs, Inc.), Bacillus stearothermophilus (Bst) DNA polymerase I (New England Biolabs, Inc.), Klenow fragment of DNA polymerase I (New England Biolabs, Inc.), M-MuLV reverse transcriptase (New England Biolabs, Inc.), VentR® (exo-minus) DNA polymerase (New England Biolabs, Inc.), VentR® DNA polymerase (New England Biolabs, Inc.), Deep Vent® (exo-) DNA polymerase (New England Biolabs, Inc.) and Bst DNA polymerase large fragment (New England Biolabs, Inc.). Where a high processivity is referred to, this typically denotes the average number of nucleotides added by a polymerase enzyme per association/dissociation with the template, i.e. the length of nascent extension obtained from a single association event.

Strand displacement-type polymerases are preferred. Preferred strand displacement-type polymerases are Phi29, Deep Vent and Bst DNA polymerase I or variants of any thereof. “Strand displacement” describes the ability of a polymerase to displace complementary strands on encountering a region of double stranded DNA during synthesis. The template is thus amplified by displacing complementary strands and synthesising a new complementary strand. Thus, during strand displacement replication, a newly replicated strand will be displaced to make way for the polymerase to replicate a further complementary strand. The amplification reaction initiates when a primer or the 3′ free end of a single stranded template anneals to a complementary sequence on a template (both are priming events). When DNA synthesis proceeds and if it encounters a further primer or other strand annealed to the template, the polymerase displaces this and continues its strand elongation. The strand displacement may release single stranded DNA which can act as a template for more priming events. The priming of the newly released DNA may lead to hyper-branching, and a high yield of products. It should be understood that strand displacement amplification methods differ from PCR-based methods in that cycles of denaturation are not essential for efficient DNA amplification, as double-stranded DNA is not an obstacle to continued synthesis of new DNA strands. Strand displacement amplification may only require one initial round of heating, to denature the initial template if it is double stranded, to allow the primer to anneal to the primer binding site, if a primer is used. Following this, the amplification may be described as isothermal, since no further heating or cooling is required. In contrast, PCR methods require cycles of denaturation (i.e. elevating temperature to 94 degrees centigrade or above) during the amplification process to melt double-stranded DNA and provide new single-stranded templates. During strand displacement, the polymerase will displace strands of already synthesised DNA. Further, it will use newly synthesised DNA as a template, ensuring rapid amplification of DNA.

A strand displacement polymerase used in a process of the invention preferably has a processivity of at least 20 kb, more preferably, at least 30 kb, at least 50 kb, or at least 70 kb or greater. In one embodiment, the strand displacement DNA polymerase has a processivity that is comparable to, or greater than phi29 DNA polymerase.

Strand displacement replication is, therefore, preferred. During strand displacement replication, the template is amplified by displacing already replicated strands, which have been synthesised by the action of the polymerase, in turn displacing another strand, which can be the original complementary strand of a double stranded template, or a newly synthesised complementary strand, the latter synthesised by the action of a polymerase on an earlier primer annealed to the template. Thus, the amplification of the template may occur by displacement of replicated strands through strand displacement replication of another strand. This process may be described as strand displacement amplification or strand displacement replication.

A preferred strand displacement replication process is Loop-mediated isothermal amplification, or LAMP. LAMP generally uses 4-6 primers recognizing 6-8 distinct regions of the template DNA. In brief, a strand-displacing DNA polymerase initiates synthesis and 2 of the primers form loop structures to facilitate subsequent rounds of amplification. An inner primer containing sequences of the sense and antisense strands of the target DNA initiates LAMP. The following strand displacement DNA synthesis primed by an outer primer releases a single-stranded DNA. This serves as template for DNA synthesis primed by the second inner and outer primers that hybridize to the other end of the target, which produces a stem-loop DNA structure. In subsequent LAMP cycling one inner primer hybridizes to the loop on the product and initiates displacement DNA synthesis, yielding the original stem-loop DNA and a new stem-loop DNA with a stem twice as long. Modified LAMP procedures can also be adopted, where fewer internal primers are required.

A preferred strand displacement replication process is rolling circle amplification (RCA). The term RCA describes the ability of RCA-type polymerases to continuously progress around a circular DNA template strand whilst extending a hybridised primer. This leads to formation of linear single stranded products with multiple repeats of amplified DNA. The sequence of the circular template (a single unit) is multiply repeated within a linear product. For a circular template, the initial product of strand displacement amplification is a single stranded concatamer, which is either sense or antisense, depending on the polarity of the template. These linear single stranded products serve as the basis for multiple hybridisation, primer extension and strand displacement events, resulting in formation of concatameric double stranded DNA products, again comprising multiple repeats of amplified DNA. There are thus multiple copies of each amplified “single unit” DNA in the concatameric double stranded DNA products. RCA polymerases are particularly preferred for use in the processes of the present invention. The products of RCA-type strand displacement replication processes may require processing to release single unit DNAs. This is desirable if single units of DNA are required. Typical strand displacement conditions using Phi29 DNA polymerase include high levels of magnesium ions, for example 10 mM magnesium (normally as a chloride salt) in combination with 0.2 to 4 mM nucleotides.

In order to allow for amplification, according to some aspects one or more primers may also be required by the enzymatic DNA synthesis. If no template is used, the primers allow for a starting point for DNA synthesis and are designed to begin the synthesis reaction. If a template is used, the primers may be non-specific (i.e. random in sequence) or may be specific for one or more sequences comprised within the template. Alternatively, a primase enzyme may be supplied to generate the primer de novo. If the primers are of random sequence they allow for non-specific initiation at any site on the template. This allows for high efficiency of amplification through multiple initiation reactions from each template strand. Examples of random primers are hexamers, heptamers, octamers, nonamers, decamers or sequences greater in length, for example of 12, 15, 18, 20 or 30 nucleotides in length. A random primer may be of 6 to 30, 8 to 30 or 12 to 30 nucleotides in length. Random primers are typically provided as a mix of oligonucleotides which are representative of all potential combinations of e.g. hexamers, heptamers, octamers or nonamers in the template.

In one embodiment, the primers or one or more of the primers are specific. This means they have a sequence which is complementary to a sequence in the template from which initiation of amplification is desired. In this embodiment, a pair of primers may be used to specifically amplify a portion of the DNA template which is internal to the two primer binding sites. Alternatively, a single specific primer may be used. A set of primers may be employed.

Primers may be any nucleic acid composition. Primers may be unlabelled, or may comprise one or more labels, for example radionuclides or fluorescent dyes. Primers may also comprise chemically modified nucleotides. For example, the primer may be capped in order to prevent initiation of DNA synthesis until the cap is removed, i.e., by chemical or physical means. Primer lengths/sequences may typically be selected based on temperature considerations i.e. as being able to bind to the template at the temperature used in the amplification step.

In certain aspects, the contacting of the template with the polymerase and one or more primers may take place under conditions promoting annealing of primers to the template. The conditions include the presence of single-stranded nucleic acid allowing for hybridisation of the primers. The conditions conventionally also include a temperature and buffer allowing for annealing of the primer to the template. Appropriate annealing/hybridisation conditions may be selected depending on the nature of the primer. An example of conventional annealing conditions, which may be used in the present invention include a buffer comprising 30 mM Tris-HCl pH 7.5, 20 mM KCl, 8 mM MgCl₂. However, the present inventors have described conditions herein with reduced buffer and divalent metal ion components that still allow for primer binding and these are discussed further below. The annealing may be carried out following denaturation using heat followed by gradual cooling to the desired reaction temperature.

However, amplification using strand displacement replication can also take place without a primer, and thus requires no hybridisation and primer extension to occur. Instead, the single stranded template self-primes by forming hairpins, which have a free 3′ end available for extension. The remaining steps of the amplification remain the same.

The template and/or polymerase are also contacted with nucleotides, as nucleotide salts. The combination of DNA template, polymerase and nucleotide salts may be described as forming a reaction mixture. The reaction mixture may also comprise one or more primers or a primase. The reaction mixture may independently also include one or more divalent metal cations. The reaction mixture may further comprise a chemical denaturant. Such denaturants can be potassium, ammonium or sodium hydroxide. The reaction mixture may further comprise additional enzymes, such as a helicase or a pyrophosphatase. The reaction mixture may contain pH buffering agents, and in some aspects, it contains no pH buffering agents.

A nucleotide is a monomer, or single unit, of nucleic acids, and nucleotides are composed of a nitrogenous base, a five-carbon sugar (ribose or deoxyribose), and at least one phosphate group. Any suitable nucleotide may be used.

The nucleotides are present as salts with monovalent cations. Monovalent cations are ionic species with a single positive charge, and thus generally up to four will be present in a nucleotide salt It is preferred that the monovalent cation has an ionic radius greater than that of the sodium ion. Ionic radius is the radius of an ion in ionic crystal structure. Ionic radii are typically given in units of either picometers (pm) or angstroms (Å). The ionic radius is not a fixed property of a given ion, but varies with various parameters including coordination number and spin state. Nonetheless, ionic radius values are sufficiently distinct to allow periodic trends to be recognized for atomic ions, with ionic radii increasing on descending a periodic table group. For the same ion, ionic radius increases with increasing coordination number, and an ion in a low-spin state will be smaller than the same ion in a high-spin state. Generally, ionic radius decreases with increasing positive charge. Thus, when ionic radius is referred to herein, it may be to any possible ionic radius of that ion. Exemplary ionic radii are presented in Table 6.

The nucleotides may include salts of monovalent metal ions, including but not limited to alkali metals (group 1): lithium (Li⁺), sodium (Na⁺), potassium (K⁺), rubidium (Rb⁺), caesium (Cs⁺) or francium (Fr⁺). Alternatively or additionally, the monovalent metal ion may be a transition metal (Group 11): copper (Cu⁺), silver (Ag⁺), gold (Au⁺) or roentgenium (Rg⁺). The alkali metals are preferred, and thus the preferred counter-ion may be potassium (K+), rubidium (Rb+), caesium (Cs+) or francium (Fr+).

The nucleotides may include salts of polyatomic monovalent ions. A polyatomic ion is an ion that contains more than one atom. This differentiates polyatomic ions from monatomic ions, which contain only one atom. Exemplary monovalent polyatomic cations include ammonium (NH₄⁺) and hydronium (H₃O⁺), wherein ammonium is particularly preferred. Ammonium has an ionic radius which is greater than sodium under all conditions. Derivatives of ammonium are also encompassed, and an exemplary list of these includes: monoalkyl ammonium, dialkyl ammonium, trialkyl ammonium, choline, quaternary ammonium and imidazolium. Those skilled in the art will be aware of further derivatives of ammonium that carry a single positive charge that are appropriate to use as a counter-ion on a nucleotide salt.

The nitrogenous base may be adenine (A), guanine (G), thymine (T), cytosine (C), and uracil (U). The nitrogenous base may also be modified bases, such as 5-methylcytosine (m5C), pseudouridine (Ψ), dihydrouridine (D), inosine (I), and 7-methylguanosine (m7G). The nitrogenous base may further be an artificial base. The concentration of nucleotide salts may include any combination of the various nitrogenous bases.

It is preferred that the five-carbon sugar is a deoxyribose, such that the nucleotide is a deoxynucleotide.

The nucleotides may be in the form of deoxynucleoside triphosphate, denoted dNTP. This is a preferred embodiment of the present invention. Suitable dNTPs may include dATP (deoxyadenosine triphosphate), dGTP (deoxyguanosine triphosphate), dTTP (deoxythymidine triphosphate), dUTP (deoxyuridine triphosphate), dCTP (deoxycytidine triphosphate), dITP (deoxyinosine triphosphate), dXTP (deoxyxanthosine triphosphate), and derivatives and modified versions thereof. It is preferred that the dNTPs comprise one or more of dATP, dGTP, dTTP or dCTP, or modified versions or derivatives thereof. It is preferred to use a mixture of dATP, dGTP, dTTP and dCTP or modified version thereof. Any suitable ratios of these dNTPs can be used, according to the needs of the reaction.

The nucleotides, or nucleotide salts, may be in solution, or may need to be supplied as a solid for example as a powder. The nucleotides, or nucleotide salts, may comprise modified nucleotides. The nucleotides, or nucleotide salts, may be provided in a mixture of one or more suitable bases, preferably, one or more of adenine (A), guanine (G), thymine (T), cytosine (C). Two, three or preferably all four nucleotides (A, G, T, and C) are used in the process to synthesise DNA. These nucleotides, or nucleotide salts, may all be present in substantially equal amounts, or more of one or two may be provided, depending on the nature of the DNA to be synthesised.

The nucleotides may all be natural nucleotides (i.e. unmodified), they may be modified nucleotides that act like natural nucleotides and are biologically active (i.e. LNA nucleotides—locked nucleic acid), they may be modified and biologically inactive or they may be a mixture of unmodified and modified nucleotides, and/or a mixture of biologically active and biologically inactive nucleotides. Each type (i.e. base) of nucleotide may be provided in one or more forms, i.e. unmodified and modified, or biologically active and biologically inactive. All of these nucleotides are capable of forming appropriate salts.

In one aspect of the invention, the nucleotides or nucleotide salts are present at a concentration of at least 10 mM. According to this aspect, the nucleotides or nucleotide salts may be present in the reaction mixture at a concentration of more than 10 mM, more than 15 mM, more than 20 mM, more than 25 mM, more than 30 mM, more than 35 mM, more than 40 mM, more than 45 mM, more than 50 mM, more than 55 mM, more than 60 mM, more than 65 mM, more than 70 mM, more than 75 mM, more than 80 mM, more than 85 mM, more than 90 mM, more than 95 mM, or more than 100 mM. Such concentrations are given as the concentration of nucleotide salt at the initiation or start of the process. The concentration is given after the addition of the nucleotide/nucleotide salts, wherein the addition may be to the reaction mixture. The nucleotide salt may be any appropriate mixture of nucleotide salts, with varying nitrogenous bases. The concentration applies to the sum total of nucleotide salts present in at the start of the process, whatever their composition. Thus, for example, a 10 mM concentration of nucleotide salts may be any mixture of dCTP, dATP, dGTP and dTTP counter-ioned with appropriate monovalent cations.

It will be understood that nucleotides supplied as salts may dissociate in water and other solvents to form an anionic nucleotide entity and a cation.

It is a preferred part of any aspect of the present invention that the nucleotide salt is formed by a counter-ion which has an ionic radius greater than that of the sodium ion. However, the polymerase or DNA synthesis enzyme is likely to tolerate some concentration of lithium and/or sodium nucleotide salts. Thus, there may be a portion of nucleotide salts included in the processes of the invention wherein the counter-ion is sodium and/or lithium. This portion is preferably less than 25%, optionally 20%, 15%, 10%, 5%, 1% or less. The polymerase or DNA synthesis enzyme may also tolerate sodium and/or lithium from other sources, such as the denaturing agent. It is preferred that the total concentration of lithium ions in the reaction mixture does not exceed 15 mM, preferably 10 mM, even more preferably 5 mM, 4 mM, 3 mM, 2 mM, 1 mM or less. Since lithium appears more inhibitory, it is preferred that this ion is substantially excluded from the reaction mixture. In the case of sodium ions, since sodium hydroxide is generally used as a denaturant, the presence of sodium ions may be tolerable.

The nucleotide salts used in the processes of the inventions may, therefore, include a mixture of different nucleotide salts, for example a mix of potassium-nucleotide salts and caesium-nucleotide salts. Any number of different salts may be used. It is preferred that at least 75% of the salts have counter-ions with an ionic radius greater than that of the sodium ion, optionally 80%, 85%, 90%, 95%, 96%, 97%, 98%, 99% or more. It may be desirable to use a mixture of different salts in order to maximise the yield of DNA and utilise the different characteristics of the various counter-ions. Alternatively written, there is provided a cell-free process for synthesising DNA comprising contacting a DNA template with at least one polymerase in the presence of one or more nucleotides in the form of a salt to form a reaction mixture, wherein said nucleotides are in the form of two or more salts, each salt comprising different monovalent cations, wherein at least one of the cations has an ionic radius greater than that of the sodium ion. Thus, two or more different nucleotide salts may be used in the processes of the invention, said salts varying by virtue of the use of a different counter-ion. It may be preferred that all of the salt requires the use of a counter-ion with an ionic radius greater than that of the sodium ion.

The enzymatic DNA synthesis may be maintained under conditions promoting synthesis of DNA, and this will depend upon the particular method selected.

Amplification of a template via strand displacement is preferred. Preferably, the conditions promote amplification of said template by displacement of replicated strands through strand displacement replication of another strand. The conditions comprise use of any temperature allowing for amplification of DNA, commonly in the range of 20 to 90 degrees centigrade. A preferred temperature range may be about 20 to about 40 or about 25 to about 35 degrees centigrade. A preferred temperature for LAMP amplification is about 50 to about 70 degrees centigrade.

Typically, an appropriate temperature for enzymatic DNA synthesis is selected based on the temperature at which a specific polymerase has optimal activity. This information is commonly available and forms part of the general knowledge of the skilled person. For example, where phi29 DNA polymerase is used, a suitable temperature range would be about 25 to about 35 degrees centigrade, preferably about 30 degrees centigrade. However, a thermostable phi29 may operate at a higher constant temperature. The skilled person would routinely be able to identify a suitable temperature for efficient amplification according to the processes of the invention. For example, a process could be carried out at a range of temperatures, and yields of amplified DNA could be monitored to identify an optimal temperature range for a given polymerase. The amplification may be carried out at a constant temperature, and it is preferred that the process is isothermal. Since strand displacement amplification is preferred there is no requirement to alter the temperature to separate DNA strands. Thus, the process may be an isothermal process.

Other conditions promoting DNA synthesis are conventionally thought to comprise the presence of suitable buffering agents/pH and other factors which are required for enzyme performance or stability. Suitable conventional conditions include any conditions used to provide for activity of polymerase enzymes known in the art.

For example, the pH of the reaction mixture may be within the range of 3 to 10, preferably 5 to 8 or about 7, such as about 7.5. pH may be maintained in this range by use of one or more buffering agents. Such buffers include, but are not restricted to MES, Bis-Tris, ADA, ACES, PIPES, MOBS, MOPS, MOPSO, Bis-Tris Propane, BES, TES, HEPES, DIPSO, TAPSO, Trizma, HEPPSO, POPSO, TEA, EPPS, Tricine, Gly-Gly, Bicine, HEPBS, TAPS, AMPD, TABS, AMPSO, CHES, CAPSO, AMP, CAPS, CABS, phosphate, citric acid-sodium hydrogen phosphate, citric acid-sodium citrate, sodium acetate-acetic acid, imidazole and sodium carbonate-sodium bicarbonate.

A buffer is generally defined by a mixture of reaction components. Usually included is a buffering agent to maintain a stable pH; one or more additional salts composed of a cationic and anionic species i.e. sodium chloride, potassium chloride; and/or detergents such as Triton-X-100 to ensure optimal activity or stability of the enzymes. A minimal buffer is composed of only a buffering reagent with no additional salts or detergents provided, with the proviso that small amounts of cationic species may be present for DNA synthesis in which chemical denaturation is required. Surprisingly, using higher concentrations of nucleotide salts in the processes of the invention permits the use of these minimal buffers.

A “no buffer” system lacks a provided or defined pH buffering agent in the mixture of reaction components and lacks additional salts or detergents. This “no-buffer” system contains only the reaction components required for the DNA synthesis alone, and contains cationic species provided for chemical denaturation or as nucleotide salt counter ions only. Thus, in this system, there are no additional ions added beyond those that serve a specific purpose in the DNA synthesis reaction. The counter-ions provided with the nucleotides (as a salt) serve to stabilise the nucleotide prior to use in the process.

While the application of heat (exposure to 95° C. for several minutes) is used to denature double stranded DNA other approaches may be used which are more suitable for DNA synthesis. Double stranded DNA can be readily denatured by exposure to a high or low pH environment or where cations are absent or present in very low concentrations, such as in deionized water. The polymerase requires the binding of a short oligonucleotide primer sequence to a single stranded region of the DNA template to initiate its replication. The stability of this interaction and therefore the efficiency of DNA synthesis may particularly be influenced by the concentration of metal cations and particularly divalent cations such as Mg²⁺ ions which may be seen as an integral part of the process.

The enzymatic DNA synthesis may also require divalent metal ions. The process may comprise the use of salts of divalent metal ions: magnesium (Mg²⁺), manganese (Mn²⁺), calcium (Ca²⁺), beryllium (Be²⁺), zinc (Zn²⁺) and strontium (Sr²⁺). The most often used divalent ions in DNA synthesis is magnesium or manganese.

The enzymatic DNA synthesis can be carried out at a lower concentration of divalent metal ions than previously thought possible. It is conventionally thought that a ratio of up to 2:1 divalent cations to nucleotides are required or optimal, and as the data in the Example shows, this holds true for nucleotide salts with lithium ions in particular which are the primarily used form. However, if alternative ions are used in these salts, the requirement for divalent ions, magnesium in particular, dramatically falls, such that the ratio of ion to nucleotide salt is in the order of 1.5:1, or about 1:1 or less. Results have even been obtained for ratios of magnesium to nucleotide salts of 0.2:1, these results being obtained with nucleotide salts with caesium. These ratios are particularly marked at higher concentrations of nucleotide salts (i.e. 20 mM or above). Therefore, the invention is also directed to the synthesis of DNA wherein the ratio of magnesium ions to nucleotide salt is 1:1 or less, characterised in that the nucleotide salt comprises a counter-ion which has an ionic radius greater than that of the sodium ion and the concentration of the nucleotide salt is more than 25 mM, more than 30 mM, more than 35 mM, more than 40 mM, more than 45 mM, more than 50 mM, more than 55 mM, more than 60 mM, more than 65 mM, more than 70 mM, more than 75 mM, more than 80 mM, more than 85 mM, more than 90 mM, more than 95 mM, or more than 100 mM.

During synthesis, polymerases release pyrophosphate from nucleotides that are incorporated into the growing DNA chain. Pyrophosphate has a binding affinity for magnesium ions similar to nucleoside triphosphate and so free magnesium ions are not released by this process. The consequence of using high starting concentrations of nucleotides during synthesis will be reduced levels of free magnesium ions. Since these ions may be required for polymerase catalytic activity, it is conventional thinking that suboptimal levels caused by interaction with phosphates or phosphate groups is likely to be detrimental to efficient amplification. Sufficient, and consequently, excess concentrations of magnesium ions were thought to be critical for DNA yield and amplification. Therefore, the ability to reduce the level of magnesium whilst maintaining yield is an exciting improvement over the art.

Accordingly, the invention provides an enzymatic DNA synthesis which is performed under conditions of a reduced ratio of divalent cations to dNTPs, comprising the use of nucleotide salts with a monovalent cation or cations with an ionic radius greater than that of the sodium ion.

The effect is particularly marked with nucleotide salts comprising ammonium and caesium, or mixtures thereof.

Detergents may also be included in the reaction mixture in certain aspects. Examples of suitable detergents include Triton X-100™, Tween20™ and derivatives of either thereof. Stabilising agents may also be included in the reaction mixture. Any suitable stabilising agent may be used, in particular, bovine serum albumin (BSA) and other stabilising proteins. Reaction conditions may also be improved by adding agents that relax DNA and make template denaturation easier. Such agents include, for example, dimethyl sulphoxide (DMSO), formamide, glycerol and betaine. DNA condensing agents may also be included in the reaction mixture. Such agents include, for example, polyethylene glycol or cationic lipid or cationic polymers.

However, in certain embodiments, these components may be reduced or removed from the reaction mixture, for example in the minimal or no-buffer systems.

It should be understood that the skilled person is able to modify and optimise synthesis conditions for the processes of the invention using these additional components and conditions on the basis of their general knowledge. Likewise the specific concentrations of particular agents may be selected on the basis of previous examples in the art and further optimised on the basis of general knowledge.

As an example, a suitable reaction buffer used in RCA-based methods in the art is 50 mM Tris HCl, pH 7.5, 10 mM MgCl₂, 20 mM (NH₄)₂SO₄, 5% glycerol, 0.2 mM BSA, 1 mM dNTPs. A preferred reaction buffer used in the RCA amplification of the invention is 30 mM Tris-HCl pH 7.9, 30 mM KCl, 7.5 mM MgCl₂, 10 mM (NH₄)₂SO₄, 4 mM DTT, 2 mM dNTPs. This buffer is particularly suitable for use with Phi29 DNA polymerase.

A suitable reaction buffer for use with the nucleotide salts of the invention is 30 mM Tris HCl, pH 7.9, 5 mM (NH₄)₂SO₄, and 30 mM KCl. Under certain circumstances, the enzymatic DNA synthesis may be conducted in water (“no buffer”).

The enzymatic DNA synthesis may also comprise the use of one or more additional proteins. The DNA template may be amplified in the presence of at least one pyrophosphatase, such as Yeast Inorganic pyrophosphatase. Two, three, four, five or more different pyrophosphatases may be used. These enzymes are able to degrade pyrophosphate generated by the polymerase from dNTPs during strand replication. Build-up of pyrophosphate in the reaction can cause inhibition of DNA polymerases and reduce speed and efficiency of DNA amplification. Pyrophosphatases can break down pyrophosphate into non-inhibitory phosphate. An example of a suitable pyrophosphatase for use in the processes of the present invention is Saccharomyces cerevisiae pyrophosphatase, available commercially from New England Biolabs, Inc.

Any single-stranded binding protein (SSBP) may be used in the processes of the invention, to stabilise single-stranded DNA. SSBPs are essential components of living cells and participate in all processes that involve ssDNA, such as DNA replication, repair and recombination. In these processes, SSBPs bind to transiently formed ssDNA and may help stabilise ssDNA structure. An example of a suitable SSBP for use in the processes of the present invention is T4 gene 32 protein, available commercially from New England Biolabs, Inc.

The yield of the reaction relates to the amount of DNA synthesised. The expected yield from a process according to the present invention may exceed 3 g/l. It is preferred that the amount of DNA synthesised is greater than 3, 4, 5, 6, 7, 8, 9, 10, 11, 12, 13, 14, 15, 16, 17, 18, 19, 20, 21, 22, 23, 24, 25, 26, 27, 28, 29 or 30 g/l or more. A preferred amount of DNA synthesised is 5 g/l. The present invention improves the yield possible from enzymatic synthesis of DNA. It is an object of the present invention to improve the yield of a cell-free enzymatic DNA synthesis process, such that DNA can be synthesised on a large scale in a cost-effective way. The present invention allows the manufacture/synthesis of DNA economically on an industrial scale using an enzymatic process catalysed by a DNA synthesis enzyme or polymerase. The present process allows the efficient incorporation of nucleotides into the DNA product. It is thought that the processes of the invention will allow reaction mixtures to be scaled up into several litres, including tens of litres. The improved yield, productivity or processivity may be compared to an identical reaction mixture where all of the nucleotides are supplied as conventional salts (sodium and/or lithium).

In one embodiment, the present invention relates to a process for enhancing the synthesis of DNA. This enhancement may be compared to an identical reaction mixture, with the exception that all of the nucleotides salts used are exclusively sodium or lithium, or a mixture thereof.

In one aspect, the invention provides a cell-free process for synthesising DNA comprising contacting a DNA template with at least one polymerase in the presence of one or more nucleotides in the form of a salt with a monovalent cation or cations to form a reaction mixture, wherein said nucleotides are present at a concentration of at least 10 mM and said cations are not exclusively sodium or lithium.

Alternatively, a cell-free process for synthesising DNA comprising contacting a DNA template with at least one polymerase in the presence of one or more nucleotides in the form of a salt to form a reaction mixture, wherein said nucleotides are present at a concentration of at least 10 mM and are either:

(a) in the form of a salt with a single monovalent cation whose ionic radius is greater than that of the sodium ion, or

(b) in the form of salts with 2 or more different monovalent cations, wherein at least one of the cations has an ionic radius greater than that of the sodium ion.

It is preferred that the concentration of nucleotides referred to herein is the starting concentration of nucleotides at the start of the process, the initial concentration when the reaction mixture is formed.

The invention may also relate to a cell-free process for synthesising DNA comprising contacting a DNA template with at least one polymerase in the presence of one or more nucleotides in the form of a salt with sodium ions in a concentration of between 10 and 20 mM, or up to 30 mM. The invention provides a cell-free process for the enzymatic synthesis of DNA comprising the use of nucleotides supplied as salts, wherein said salts comprise a monovalent cation with an ionic radius that is greater than that of the sodium ion, preferably wherein the nucleotide salts are supplied or are present in a concentration greater than 10 mM.

The invention further provides an enzymatic DNA synthesis which is performed under conditions of reduced divalent cations, preferably magnesium, comprising the use of nucleotide salts with a monovalent cation or cations with an ionic radius greater than that of the sodium ion.

The invention may alternatively be performed using nucleotide salts with a counter-ion which has an ionic radius greater than that of the potassium ion, optionally wherein the concentration of said nucleotide salt is more than 25 mM, more than 30 mM, more than 35 mM, more than 40 mM, more than 45 mM, more than 50 mM, more than 55 mM, more than 60 mM, more than 65 mM, more than 70 mM, more than 75 mM, more than 80 mM, more than 85 mM, more than 90 mM, more than 95 mM, or more than 100 mM.

Nucleotides in the form of salts are also referred to herein as nucleotide salts.

The invention will now be described with reference to several non-limiting examples.

### EXAMPLES

### Materials and Methods

### Reagents

The following reagents were used in the presented examples:

dNTP salts lithium salt, stock concentration 100 mM (Bioline)

dNTP salts sodium, potassium, caesium, ammonium, salts, stock concentration 100 mM (Contract synthesis)

Phi29 DNA polymerase, stock concentration 2.4 g/l (produced in-house)

Thermostable pyrophosphatase, stock concentration 2000 U/ml (Enzymatics)

DNA primer, stock concentration 5 mM (Oligofactory)

Plasmid template: ProTLx-K B5X4 LUX 15-0-15-10-15 AT-STEM, stock concentration 0.1 g/l (produced in house)

Nuclease free water (Sigma Aldrich)

Magnesium chloride, stock concentration 2M (Sigma Aldrich)

Tris-base (Thermo Fisher Scientific)

Tris-HCl (Sigma Aldrich)

NaCl (Sigma Aldrich)

EDTA, stock concentration 0.5 M (Sigma Aldrich)

PEG 8000 (Applichem)

Ethanol (Thermo Fisher Scientific)

GeneRuler 1 kb+DNA ladder (Thermo Fisher Scientific)

TAE buffer from 20× stock (Thermo Fisher Scientific)

Potassium chloride (Sigma Aldrich)

Lithium chloride (Sigma Aldrich)

Caesium chloride (Sigma Aldrich)

Ammonium chloride (Sigma Aldrich)

Ammonium sulphate (Thermo Fisher Scientific)

### Example 1

Rolling Circle Amplification (RCA) Reactions at Different Concentrations of Magnesium Ions and Nucleotide Salts (dNTP Salts) (Counter-Ioned with Lithium, Sodium, Potassium, Caesium and Ammonium Cations); Effect on DNA Yield.

### INTRODUCTION

The concentration of magnesium ions in a reaction buffer is critical for optimal synthesis of DNA by a DNA polymerase. It has been reported that low magnesium ion concentrations can result in the synthesis of little or no DNA while high concentrations often result in the production of non-specific products as well as causing mis-incorporation of dNTPs and subsequent increase in replication errors. Since magnesium binds to phosphate moieties of each dNTP, common practice is to use a concentration of magnesium ions equivalent to or higher than the concentration of dNTPs used (Dean, F. B., Nelson, J. R., Giesler, T. L., & Lasken, R. S. (2001). Rapid Amplification of Plasmid and Phage DNA Using Phi29 DNA Polymerase and Multiply-Primed Rolling Circle Amplification. Genome Research, 11(6), 1095-1099. http://doi.org/10.1101/gr.180501). Magnesium-dNTP is an absolute requirement for high fidelity DNA synthesis by the DNA polymerase. Magnesium also bind to DNA and can effect structural changes and form cross links between separate strands at concentrations higher than that required for DNA synthesis.

For the enzymatic production of DNA in industrially relevant quantities, it is necessary to maximise the concentration of dNTPs used in a reaction in order to achieve the highest yields of DNA. In addition, the reaction needs to be both efficient and accurate. The dNTPs available commercially are either sodium or lithium salts typically with 4 metal monovalent cations per molecule. Most publications on DNA synthesis ignore the nature of the counter-ion and their possible influence on the formation of magnesium-dNTPs. If the concentrations of dNTPs are increased in a reaction, the concentration of the monovalent counter-ions can increase by factor of 4 so they have a potential impact on the DNA amplification reaction.

Understanding the magnesium-monovalent counter-ion dynamics in DNA synthesis is therefore critical for maximising DNA yield at the lowest possible magnesium concentration to allow highest accuracy of the DNA product.

The following set of experiments assessed the impact of increasing initial magnesium concentrations (5 mM, 10 mM, 20 mM and 40 mM) and different salts of dNTPs on the yields of DNA amplified by RCA (Rolling Circle Amplification).

### Reaction Setup

Reactions were set up at 100 μl scale as follows: A denaturation mix was prepared, and left at room temperature while the reaction mix was assembled. These were then mixed, and the DNA polymerase and pyrophosphatase added. Table 1 shows the experimental protocol.

RCA reactions were incubated at 30° C. for a minimum of 48 hours before processing.

TABLE 1
RCA reaction components

| | | Stock | | Reaction |
|---|---|---|---|---|
| | Reagent | concentration | Volume | concentration |
| Denaturation | Plasmid | 0.1 g/l | 0.5 ul | 0.5 ng/μl |
| mix | Template | | | |
| | NaOH | 1 M | 0.5 μl | 5 mM |
| | DNA primer | 5 mM | 1 μl | 50 μM |
| | H₂O | | 3 μl | to 5 μl |
| Reaction | 10 × Tris buffer: | | 10 μl | 30 mM |
| mix | 300 mM | | | |
| | Tris, pH 7.9 | | | 30 mM |
| | 300 mM KCl | | | 5 mM |
| | 50 mM | | | |
| | (NH₄)₂SO₄ | | | |
| | MgCl₂ | 2 M | Variable | Variable—see |
| | | | | data |
| | dNTP salts | 100 mM | Variable | Variable—see |
| | | | | data |
| | H₂O | | Variable | to 100 μl total |
| Enzyme 1 | Phi29 DNA | 2.4 g/l | 0.2 μl | 4.8 μg/ml |
| | polymerase | | | |
| Enzyme 2 | Pyrophosphatase | 2000 U/ml | 0.1 μl | 0.2 U |

### Sample Processing Procedure

A 1.5× molar excess of EDTA to MgCl₂ was added after 48 hrs of RCA and reactions were brought to 800 μl volume with water. They were shaken vigorously for 15 minutes and placed on a rotator until the reaction was fully mixed. Reactions were then taken to 1 ml in 1M NaCl by addition of 200 μl 5M NaCl. Concatemeric DNA was then precipitated by further addition of 100 μl 50% (w/v) PEG 8000. The mixtures were shaken vigorously for 15 minutes to ensure full precipitation and then spun at 13,000 rpm in a bench-top centrifuge for 10 minutes. The supernatants were carefully decanted, and the pellets washed with 500 μl 100% ethanol. Pellets were re-centrifuged at 13,000 rpm in a bench-top centrifuge for 10 minutes and the ethanol supernatant carefully decanted. The pellets were left to dry for 5 minutes to evaporate residual ethanol, re-suspended in 1 ml water and placed on a rotator overnight.

Reaction DNA concentrations were quantified from UV absorption measurements using an Implen NP80 nanophotometer. Data is corrected for the 10× fold increase in reaction volume and concentrations are expressed in g/l of original volume vs dNTP concentrations used.

### Results

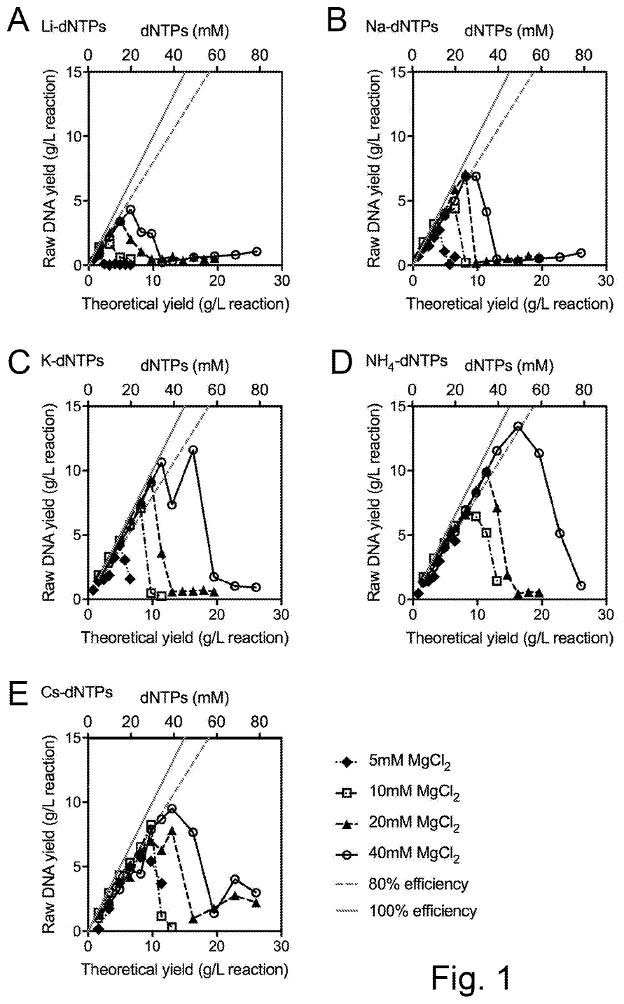

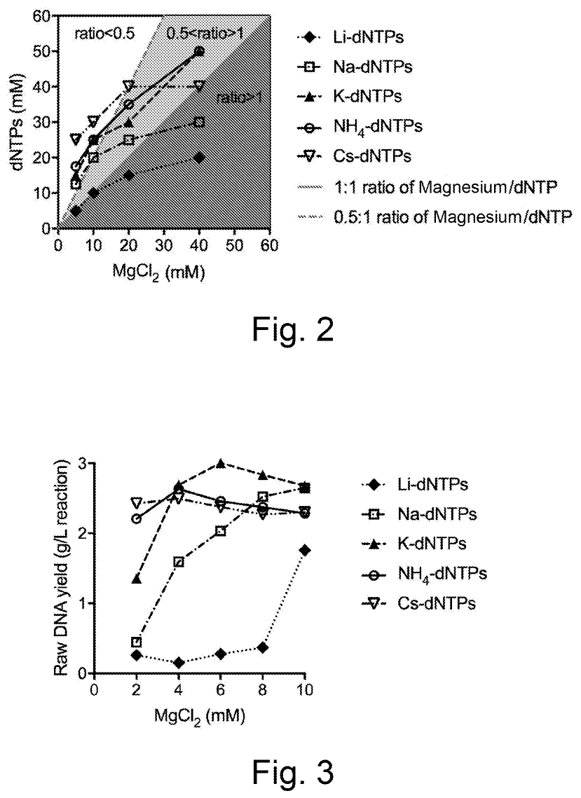

Tables 2 to 5 and FIGS. 1 and 2 show that the initial concentrations of magnesium and starting concentrations of different dNTP salts affect raw DNA yields. Values in brackets denote the ratio of magnesium/dNTP at the highest DNA yield achieved for each type of dNTP salt.

TABLE 2
Reaction concentration of 5 mM MgCl₂. Peak yields are highlighted
in bold, and numbers in brackets are magnesium/dNTP ratio:

| Starting | | | | | |
|---|---|---|---|---|---|
| concentration | Raw DNA yield (g/l) | | | | |
| dNTP salts | lithium- | sodium- | potassium- | ammonium- | caesium- |
| (mM) | dNTPs | dNTPs | dNTPs | dNTPs | dNTPs |
| 2.5 | 0.469 | 0.678 | 0.072 | 0.451 | — |
| 5 | 0.914 | 1.312 | 1.392 | 1.328 | 0.166 |
| | (1.0) | | | | |
| 7.5 | 0.096 | 1.482 | 1.52 | 1.432 | — |
| 10 | 0 | 2.16 | 1.852 | 1.768 | 1.733 |
| 12.5 | 0.098 | 2.728 | 3.24 | 2.971 | — |
| | | (0.40) | | | |
| 15 | 0.047 | 1.084 | 4.172 | 3.98 | 3.719 |
| | | | (0.33) | | |
| 17.5 | 0.057 | 0.056 | 3.056 | 5.096 | — |
| | | | | (0.29) | |
| 20 | 0.033 | 0.647 | 1.58 | 4.536 | 4.953 |
| 25 | — | — | — | — | 5.719 |
| | | | | | (0.20) |
| 30 | — | — | — | — | 5.425 |
| 35 | — | — | — | — | 3.689 |

TABLE 3
Reaction concentration of 10 mM MgCl₂. Peak yields are
highlighted in bold and numbers in brackets are magnesium/dNTP ratio:

| Starting | | | | | |
|---|---|---|---|---|---|
| concentration | Raw DNA yield (g/l) | | | | |
| dNTP salts | lithium- | sodium- | potassium- | ammonium- | caesium- |
| (mM) | dNTPs | dNTPs | dNTPs | dNTPs | dNTPs |
| 5 | 1.405 | 1.820 | 1.900 | 1.740 | 1.450 |
| 10 | 1.661 | 3.201 | 3.310 | 3.210 | 2.981 |
| | (1.0) | | | | |
| 15 | 0.604 | 4.010 | 4.571 | 4.366 | 4.340 |
| 20 | 0.487 | 4.407 | 5.777 | 5.730 | 5.320 |
| | | (0.50) | | | |
| 25 | — | 0.200 | 7.071 | 6.872 | 6.529 |
| | | | (0.40) | (0.40) | |
| 30 | — | — | 0.488 | 6.448 | 8.262 |
| | | | | | (0.33) |
| 35 | — | — | 0.232 | 5.164 | 1.160 |
| 40 | — | — | — | 1.441 | 0.301 |

TABLE 4
Reaction concentration 0f 20 mM MgCl₂. Peak yields are highlighted
in bold and numbers in brackets are magnesium/dNTP ratio:

| Starting | | | | | |
|---|---|---|---|---|---|
| concentration | Raw DNA yield (g/l) | | | | |
| dNTP salts | lithium- | sodium- | potassium- | ammonium- | caesium- |
| (mM) | dNTPs | dNTPs | dNTPs | dNTPs | dNTPs |
| 5 | 1.501 | 1.66 | 1.8165 | 1.773 | 1.243 |
| 10 | 2.529 | 3.011 | 3.114 | 2.955 | 2.384 |
| 15 | 3.488 | 4.1 | 4.6275 | 4.364 | 3.775 |
| | (1.33) | | | | |
| 20 | 2.02 | 5.892 | 6.3015 | 5.569 | 4.178 |
| 25 | 1.062 | 7.105 | 7.5735 | 6.566 | 6.225 |
| | | (0.80) | | | |
| 30 | 0.475 | 0.168 | 9.285 | 8.495 | 6.973 |
| | | | (0.67) | | |
| 35 | 0.491 | 0.318 | 3.6015 | 10.11 | 6.278 |
| | | | | (0.57) | |
| 40 | 0.683 | 0.35 | 0.588 | 7.131 | 7.803 |
| | | | | | (0.50) |
| 45 | 0.385 | 0.541 | 0.6255 | 1.921 | — |
| 50 | 0.655 | 0.494 | 0.639 | 0.449 | 0.978 |
| 55 | 0.466 | 0.718 | 0.723 | 0.576 | — |
| 60 | 0.54 | 0.638 | 0.5955 | 0.549 | 1.799 |
| 70 | — | — | — | — | 2.762 |
| 80 | — | — | — | — | 2.224 |

TABLE 5
Reaction concentration 0f 40 mM MgCl₂. Peak yields are highlighted
in bold and numbers in brackets are magnesium/dNTP ratio:

| Starting | | | | | |
|---|---|---|---|---|---|
| concentration | Raw DNA yield (g/l) | | | | |
| dNTPsalts | lithium- | sodium- | potassium- | ammonium- | caesium- |
| (mM) | dNTPs | dNTPs | dNTPs | dNTPs | dNTPs |
| 5 | 0.791 | 1.346 | 1.4895 | 1.341 | 0.978 |
| 10 | 2.178 | 2.338 | 2.7938 | 2.533 | 2.053 |
| 15 | 3.386 | 3.847 | 4.35 | 4.035 | 3.218 |
| 20 | 4.328 | 4.992 | 5.64 | 5.245 | 4.725 |
| | (2.0) | | | | |
| 25 | 2.565 | 6.881 | 7.389 | 6.961 | 4.449 |
| 30 | 2.458 | 6.897 | 9.063 | 8.275 | 7.887 |
| | | (1.3) | | | |
| 35 | 0.231 | 4.145 | 10.647 | 9.865 | 8.669 |
| 40 | 0.326 | 0.475 | 7.3365 | 11.549 | 9.500 |
| | | | | | (1.0) |
| 50 | 0.604 | 0.315 | 11.60 | 13.44 | 7.659 |
| | | | (0.80) | (0.80) | |
| 60 | 0.691 | 0.519 | 1.743 | 11.35 | 1.375 |
| 70 | 0.8075 | 0.617 | 1.032 | 5.119 | 4.013 |
| 80 | 1.075 | 0.962 | 0.93 | 1.071 | 2.986 |

The data in Tables 2 to 5 demonstrate that the highest yields of DNA are achieved with non-commercially available potassium, ammonium and caesium dNTP salts. By using these dNTP salt counter-ions and increasing the magnesium concentration to 40 mM it is possible to use starting concentrations of up to 50 mM dNTPs and achieve efficient conversion into DNA by Phi29 DNA polymerase.

Lithium-dNTPs were poor substrates for DNA synthesis requiring much higher levels of magnesium than the other monovalent cations. Indeed, the peak DNA yield at 40 mM magnesium (4.328 g/l) occurred at a concentration of only 20 mM dNTPs. Sodium-dNTPs performed better than the lithium equivalents with a peak DNA yield (6.897 g/l) at 40 mM magnesium achieved with 30 mM dNTPs.

Ammonium was the best dNTP counter-ion to achieve the highest DNA yield (13.44 g/l) at the highest starting concentrations of dNTPs (50 mM dNTPs and 40 mM MgCl₂) while maintaining a magnesium/dNTP ratio of 0.8. The trend in the data indicates that it should be possible to further increase the starting concentration of ammonium-dNTPs and their incorporation into DNA by further increasing the MgCl₂ concentration.

Potassium-dNTPs were also superior to their lithium and sodium counterparts in both the yield of DNA achieved at high dNTP concentration (50 mM and 40 mM MgCl₂) and a magnesium/dNTP ratio of 0.80. Under the reaction conditions, potassium-dNTPs performed almost comparably to ammonium-dNTPs.

At 5 mM and 10 mM MgCl₂ in the process, the highest DNA yields (5.719 g/l and 8.262 g/l) are achieved with caesium-dNTPs at a starting concentration of 25 mM and 30 mM, respectively. The magnesium/dNTP ratio at 5 mM MgCl₂ and 25 mM dNTPs was 0.2 and the lowest recorded for all the presented data. The use of caesium-dNTPs is therefore advantageous under conditions when it is beneficial (to the outcome of the DNA amplification process) to use the lowest possible concentrations of magnesium ions, while still producing high yields.

The ammonium ion is unique amongst the other mono-cations investigated in that it is polyatomic and is completely non-metallic. It can act as a pH buffer and at its pKₐ of 9.24 it exists as 50% ammonia (NH₃) in water. The volatility of NH₃ allows for the use of DNA processing techniques, like evaporation under low pressure, not possible with metal mono-cations.

FIG. 1 is a graphical representation of the data shown in Tables 2 to 5 and shows plots of the obtained raw DNA yield (g/l) versus the theoretical DNA yield (g/l) corresponding to the total initial/starting nucleotide salt concentration (mM) at different concentrations of magnesium chloride.

FIG. 2 is a plot of dNTP salt concentration (mM) that gave maximum raw DNA yield against different reaction concentrations of magnesium ions. It clearly shows that the dependency for magnesium is highest for lithium-dNTPs and sodium-dNTP but is reduced for the other counter-ions.

Table 6 includes the ionic radii of the monovalent counter-ions at different coordination numbers. There is a clear relationship between the size of the counter-ion (relative to magnesium) and the concentration of magnesium required for utilisation of high levels of dNTPs. Larger cations such as potassium, caesium and ammonium are far superior to sodium and particularly lithium.

TABLE 6
Atomic radii of counter-ions:

| Coordination | Lithium | Sodium | Potassium | Rubidium | Caesium | Ammonium | Magnesium |
|---|---|---|---|---|---|---|---|
| number | Ionic radii (pm) | | | | | | |
| 4 | 59 | 99 | 137 | | | 140 | 57 |
| 5 | | 100 | | | | | 66 |
| 6 | 76 | 102 | 138 | 152 | 167 | 148 | 72 |
| 7 | | 112 | 146 | 156 | | | |
| 8 | 92 | 118 | 151 | 161 | 174 | 154 | 89 |
| 9 | | 124 | 155 | 163 | 178 | | |
| 10 | | | 159 | 166 | 181 | | |
| 11 | | | | 169 | 185 | | |
| 12 | | 139 | 164 | 172 | 188 | 167 | |
| 13 | | | | | | | |
| 14 | | | | 183 | | | |
| Average | 76 | 113 | 150 | 165 | 179 | 152 | 71 |
| % increase | 7 | 60 | 111 | 133 | 152 | 114 | — |
| in size over | | | | | | | |
| magnesium | | | | | | | |

References: http://abulafia.mt.ic.ac.uk/shannon/ptable.php, Shriver & Atkins

Thus, by the selective use of dNTP salt counter-ions, DNA yields in industrial processes can be increased. This may be mediated by differential affinity of the counter-ions for dNTPs, DNA, and released phosphate (PO₄³⁻) anions and the competitive dynamics with magnesium divalent cations as demonstrated in Example 6.

### Example 2

Rolling Circle Amplification (RCA) Reactions at Different Concentrations of Magnesium Ions and a Fixed Concentration of dNTP Salts (Counter-Ioned with Lithium, Sodium, Potassium, Caesium and Ammonium Cations); Effect on DNA Yield.

### Introduction & Reaction Setup

This experiment was designed to determine the minimum concentration of magnesium ions required for the incorporation of a fixed amount of dNTPs (10 mM) in the process at the start. RCA reactions and processing were performed as described in Example 1. Concentrations of dNTPs (as lithium, sodium, potassium, caesium and ammonium salts) were fixed at 10 mM in the process at the start and reactions were performed in standard RCA buffer used for Example 1 supplemented with 2 mM, 4 mM, 6 mM, 8 mM, and 10 mM MgCl₂.

### Results

The results show that, in contrast to the wide held view that a ratio of at least 1:1 of magnesium/dNTP is required for efficient dNTP incorporation, when changing to dNTPs with different counter-ions than lithium, it is possible to decrease magnesium levels in the RCA reaction well below this ratio while additionally improving the DNA yield.

As can be seen from FIG. 3 yields for both sodium-dNTPs and lithium-dNTPs have a strong dependence on magnesium levels. Although there is a slight reduction in DNA produced at 2 mM MgCl₂ for potassium-dNTPs, this form of dNTP salt along with caesium-dNTPs and ammonium-dNTPs show a lower dependency on magnesium ion concentration. This suggests that the general assumption of an optimal magnesium/dNTP ratio of 1:1 is misleading without taking into account the type of dNTP counter-ion. The data shows that the use of alternative counter-ions to lithium and sodium can reduce this ratio as low as 0.2:1.

### Example 3

Rolling Circle Amplification (RCA) Reactions in a Minimal Buffer at a Fixed Level of Magnesium Ions and Increasing Concentrations of dNTP Salts (Counter-Ioned with Lithium, Sodium, Potassium, and Ammonium Cations); Effect on DNA Yield.

### Introduction & Reaction Setup

In order to remove possible counter-ion effects of buffer components, experiments were next performed in a minimal buffer consisting of solely 30 mM Tris HCl (pH7.9) supplemented with 5 mM MgCl₂. These reactions examined the effects of increasing starting dNTP salt concentrations (from 2.5 mM to 20 mM dNTPs supplied as lithium, sodium, potassium, or ammonium salts) in reactions containing 5 mM MgCl₂.

TABLE 7
RCA reaction components with minimal buffer:

| | | Stock | | Final reaction |
|---|---|---|---|---|
| | Reagent | concentration | Volume | concentration |
| Denaturation | Plasmid Template | 0.1 g/l | 0.5 μl | 0.5 ng/μl |
| mix | NaOH) | 1 M | 0.5 μl | 5 mM |
| | DNA primer | 5 mM | 1 μl | 50 μM |
| | H₂O | | 3 μl | to 5 μl |
| Reaction | 10 × Tris buffer: | | 10 μl | 30 mM |
| mix | 300 mM Tris, pH 7.9 | | | |
| | MgCl₂ | 2 M | 0.25 μl | 5 mM |
| | dNTP salts | 100 mM | Variable | Variable—as |
| | | | | shown |
| | H₂O | | Variable | to 100 μl total |
| Enzyme 1 | Phi29 DNA | 2.4 g/l | 0.2 μl | 4.8 μg/ml |
| | polymerase | | | |
| Enzyme 2 | Pyrophosphatase | 2000 U/ml | 0.2 μl | 0.2 U |

DNA processing and quantification was performed as described in Example 1.

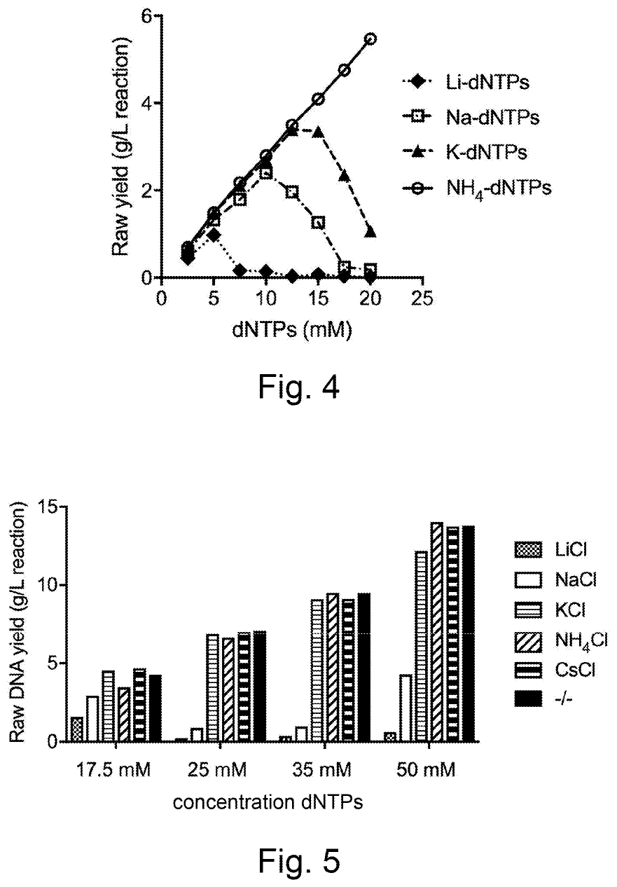

Results are shown in FIG. 4.

The data shows that RCA proceeds without the presence of 30 mM KCl and 5 mM (NH₄)₂SO₄ present in the standard reaction buffer. The observed trend in yield increase vs dNTP salt concentration with variable counter-ions is consistent with the data presented in Example 1, identifying ammonium-dNTPs as performing better than the other counter-ioned dNTP salts.

FIG. 4 validates that changing the dNTP salt counter-ion enables the RCA to proceed at higher concentrations of dNTPs, with a corresponding increase in yield.

TABLE 8
Raw DNA yield from various counter-ioned dNTPs performed in
minimal buffer at 5 mM MgCl₂. Peak yields are highlighted in
bold the ratio of magnesium/dNTPs is shown in brackets:

| Starting | | | | |
|---|---|---|---|---|
| concentration | Raw DNA yield (g/l) | | | |
| dNTP salts | lithium- | sodium- | potassium- | ammonium- |
| (mM) | dNTPs | dNTPs | dNTPs | dNTPs |
| 2.5 | 0.448 | 0.608 | 0.655 | 0.69 |
| 5 | 0.975 | 1.326 | 1.472 | 1.487 |
| | (1.0) | | | |
| 7.5 | 0.164 | 1.79 | 2.112 | 2.176 |
| 10 | 0.145 | 2.403 | 2.697 | 2.793 |
| | | (0.50) | | |
| 12.5 | 0.031 | 1.961 | 3.393 | 3.496 |
| | | | (0.40) | |
| 15 | 0.072 | 1.266 | 3.355 | 4.094 |
| 17.5 | 0.036 | 0.242 | 2.362 | 4.755 |
| 20 | 0.019 | 0.181 | 1.071 | 5.473 |
| | | | | (0.25) |

### Example 4

### Rolling Circle Amplification (RCA) Reactions at Different Concentrations of Magnesium Ions and Ammonium-dNTPs to Determine Highest Raw DNA Yield

### Introduction & Reaction Setup

These reactions aimed to extend the experimental data shown in Example 3 (FIG. 4), and find the limits of DNA yield by increasing the concentration of ammonium-dNTPs in the presence of different magnesium concentrations. RCA reactions and DNA processing were performed in a minimal buffer essentially as described in Example 3.

### Results

TABLE 9
Raw DNA yield from ammonium-dNTPs performed in minimal
buffer at different MgCl₂ concentrations. Peak yields are highlighted
in bold, and numbers in brackets is the ratio of magnesium/dNTP:

| | Raw DNA yield (g/l) | | | |
|---|---|---|---|---|
| Starting | 5 mM | 5 mM | | |
| concentration | MgCl₂ | MgCl₂ | | |
| ammonium- | (first | (second | 40 mM | 80 mM |
| dNTPs (mM) | run) | run) | MgCl₂ | MgCl₂ |
| 2.5 | 0.69 | — | — | — |
| 5 | 1.487 | — | — | — |
| 7.5 | 2.176 | — | — | — |
| 10 | 2.793 | 2.671 | — | — |
| 12.5 | 3.496 | — | — | — |
| 15 | 4.094 | 4.146 | — | — |
| 17.5 | 4.755 | — | — | — |
| 20 | 5.473 | 5.585 | — | — |
| | (0.25) | | | |
| 25 | — | 6.001 | — | — |
| | | (0.20) | | |
| 30 | — | 2.826 | — | — |
| 35 | — | 2.111 | — | — |
| 40 | — | — | 10.083 | 9.125 |
| 50 | — | — | 14.23 | 11.342 |
| | | | (0.80) | |
| 60 | — | — | 13.247 | 14.716 |
| 70 | — | — | 0.267 | 15.473 |
| 80 | — | — | 1.229 | 16.34 |
| | | | | (1.0) |

The data demonstrate that by using ammonium counter-ioned dNTPs, it is possible to further increase the starting concentration of dNTPs in a reaction (up to 80 mM) and produce very high levels of DNA. This is achieved by significantly increasing concentrations of MgCl₂ to 80 mM in a minimal buffer. Even at 80 mM MgCl₂ and 80 mM ammonium-dNTPs it is evident that the peak yield of DNA has not been reached. Adding more dNTPs should increase the DNA yield even further. Increased concentrations (beyond 80 mM) of MgCl₂ and ammonium-dNTPs should yield even higher levels of DNA under conditions where the magnesium/dNTP ratios would be expected to be <1.

### Example 5

### Determination of Productivity Limits of RCA in Water-Magnesium Chloride Mix.

### Introduction & Reaction Setup

DNA amplification experiments were then performed at 10 mM, 20 mM and 40 mM concentrations of MgCl₂ with a range of potassium-, caesium- and ammonium-dNTPs in a reaction medium containing no Tris buffering agent or other salts conventionally necessary for optimal DNA amplification. Lithium and sodium-dNTPs were omitted at that point, as both were outperformed by the other cations in the screen. Apart from the magnesium and the dNTP counter-ions, the only other cations in the reaction comprised 5 mM sodium ions from the NaOH used for template denaturation. The experiment was carried out to determine whether the dNTPs themselves and the phosphate by-product of the reaction were capable of maintaining the pH at a level that facilitated Phi29 DNA polymerase activity and the physico-chemical conditions needed for effective DNA priming.

### Results

TABLE 10
RCA reaction components with no Tris buffering agent:

| | | Stock | | Final reaction |
|---|---|---|---|---|
| | Reagent | concentration | Volume | concentration |
| Denaturation | Plasmid | 0.1 g/l | 0.5 μl | 0.5 ng/μl |
| mix | Template | | | |
| | NaOH) | 1 M | 0.5 μl | 5 mM |
| | DNA primer | 5 mM | 1 μl | 50 μM |
| | H₂O | | 3 μl | to 5 μl |
| Reaction | MgCl₂ | 2 M | Variable | Variable |
| mix | dNTP salts | 100 mM | Variable | Variable- |
| | | | | as shown |
| | H₂O | | Variable | to 100 μl total |
| Enzyme 1 | Phi29 DNA | 2.4 g/l | 0.2 μl | 4.8 μg/ml |
| | polymerase | | | |
| Enzyme 2 | Pyrophosphatase | 2000 U/ml | 0.2 μl | 0.2 U |

DNA processing and quantification was performed essentially as described in Example 1.

TABLE 11
No buffer medium with 10 mM MgCl₂. Peak yields are highlighted in
bold, and numbers in brackets are magnesium/dNTP ratio:

| Starting | | | |
|---|---|---|---|
| concentration | Raw DNA yield (g/l) | | |
| dNTP salts | potassium- | caesium- | ammonium- |
| (mM) | dNTPs | dNTPs | dNTPs |
| 5 | 0.113 | 0.067 | 1.575 |
| 10 | 0.271 | 0.16 | 3.111 |
| 15 | 0.284 | 1.456 | 4.618 |
| 20 | 0.577 | 1.64 | 5.691 |
| 25 | 0.682 | 3.220 | 6.356 |
| | (0.40) | (0.40) | (0.40) |
| 30 | 0.579 | 1.966 | 6.333 |
| 35 | 0.545 | 0.738 | 5.671 |
| 40 | 0.617 | 0.698 | 3.561 |

TABLE 12
No buffer medium with 20 mM MgCl₂. Peak
yields are highlighted in bold, and numbers
in brackets are magnesium/dNTP ratio:

| Starting | | |
|---|---|---|
| concentration | Raw DNA yield (g/l) | |
| dNTP salts | caesium- | ammonium- |
| (mM) | dNTPs | dNTPs |
| 5 | 0.128 | 1.362 |
| 10 | 2.62 | 2.941 |
| 15 | 4.18 | 4.575 |
| 20 | 5.79 | 6.153 |
| 25 | 7.710 | 8.019 |
| | (0.28) | |
| 30 | 7.428 | 9.391 |
| 35 | 7.438 | 11.60 |
| | | (0.60) |
| 40 | 6.53 | 11.592 |
| 50 | 2.211 | 5.042 |

TABLE 13
No buffer medium with 40 mM MgCl₂. Peak yields are highlighted
in bold, and numbers in brackets are [Mg]/[dNTPs] ratio:

| Starting | | | |
|---|---|---|---|
| concentration | Raw DNA yield (g/l) | | |
| dNTP salts | potassium- | caesium- | ammonium- |
| (mM) | dNTPs | dNTPs | dNTPs |
| 5 | 0.095 | 0.126 | 1.013 |
| 10 | 0.103 | 2.25 | 2.338 |
| 15 | 3.6 | 2.962 | 3.078 |
| 20 | 0.858 | 4.994 | 5.137 |
| 25 | 3.325 | 6.117 | 6.843 |
| 30 | 7.31 | 7.321 | 8.56 |
| | (1.33) | | |
| 35 | 2.296 | 6.663 | 10.205 |
| 40 | 0.369 | 7.47 | 11.454 |
| 50 | — | 11.21 | 10.684 |
| | | (0.80) | |
| 60 | — | 10.925 | 11.76 |
| | | | (0.67) |
| 70 | — | 2.539 | 1.478 |

The experimental data shows that the reactions carried out with potassium-dNTPs gave variable results in the absence of Tris buffer. On the other hand, both caesium-dNTPs and ammonium-dNTPs gave progressively higher DNA yields with increasing concentrations of magnesium ions and dNTPs. Caesium-dNTPs performed significantly better under these unbuffered conditions compared to the standard buffered environment (see Table 5). High DNA yields were recorded at 40 mM MgCl₂ and 50 mM caesium-dNTPs (magnesium/dNTP ratio of 0.80). There was no significant difference between the DNA yield using ammonium-dNTPs in buffered or unbuffered conditions. A high DNA yield was observed at 40 mM MgCl₂ with 60 mM ammonium-dNTPs (magnesium/dNTP ratio of 0.67).

### Example 6

### Effect of Other Counter-Ion Salts on DNA Amplification Using AMMONIUM-dNTPs

### Introduction & Reaction Setup

This experiment was carried out to demonstrate the effect of lithium, sodium and potassium cations on the yield of DNA obtained by RCA using ammonium-dNTPs.

TABLE 14
Reaction components:

| | | Stock | | Final reaction |
|---|---|---|---|---|
| | Reagent | concentration | Volume | concentration |
| Denaturation | Plasmid Template | 0.1 g/l | 0.5 μl | 0.5 ng/μl |
| mix | NaOH | 1 M | 0.5 μl | 5 mM |
| | DNA primer | 5 mM | 1 μl | 50 μM |
| | H₂O | | 3 μl | to 5 μl |
| Reaction mix | Tris buffer | 10 × (300 mM | 10 μl | 30 mM |
| | | Tris) | | |
| | MgCl₂ | 2 M | variable | Variable—as |
| | | | | shown |
| | ammonium- | 100 mM | variable | Variable—as |
| | dNTPs | | | shown |
| | Counter-ion salts | 1 M | variable | Variable, at 1:1 |
| | (LiCl, NaCl, | | | ratio with |
| | KCl or NH₄Cl) | | | dNTP derived |
| | | | | counter-ions |
| | H₂O | | variable | to 100 μl total |
| Enzyme 1 | Phi29 DNA | 2.4 g/l | 0.2 μl | 4.8 μg/ml |
| | polymerase | | | |
| Enzyme 2 | Pyrophosphatase | 2000 U/ml | 0.2 μl | 0.4 U |

The reactions were set up as indicated in Table 14. Four groups of experiments were conducted containing starting concentrations of 17.5 mM, 25 mM, 35 mM and 50 mM ammonium-dNTPs with 5 mM, 10 mM, 20 mM and 40 mM MgCl₂ respectively. To each group LiCl, NaCl, KCl, or NH₄Cl was added at total concentrations of 70 mM, 100 mM, 140 mM and 200 mM respectively. This resulted in an additional cation concentration in competition with the dNTP ammonium counter-ion concentrations. Furthermore, the concentration of ammonium was doubled when adding NH₄Cl. The magnesium/dNTP ratio for each group of experiments is below 1.0.

DNA processing and quantification was performed essentially as described for Example 1.

Results are shown in FIG. 5.

FIG. 5 shows that caesium, ammonium and potassium ions are not inhibitory to the synthesis of DNA when using ammonium-dNTPs. Furthermore, the ammonium concentration can even be doubled without affecting the DNA yield.

On the contrary, lithium and sodium are inhibitory, with lithium being more inhibitory than sodium. On this basis, the presence of lithium and sodium should be avoided in industrial DNA production processes requiring high concentrations of dNTPs for high DNA yields.

### Example 7

Investigation of the Buffering of DNA Synthesis Reactions by dNTPs

### Introduction & Reaction Setup

This experiment was carried out to look at the ability of dNTP salts to buffer a reaction mixture in the absence of any specific buffering agents.

TABLE 15
Experimental set-up for pH measurements in 10 mM MgCl₂

| | [dNTP] (mM) | | | | | | | | | |
|---|---|---|---|---|---|---|---|---|---|---|
| components | 5 | 10 | 15 | 20 | 25 | 30 | 35 | 40 | 50 | 60 |
| 5 mM NaOH | 0.25 | 0.25 | 0.25 | 0.25 | 0.25 | 0.25 | 0.25 | 0.25 | 0.25 | 0.25 |
| (μl) | | | | | | | | | | |
| H₂O (μl) | 47 | 44.5 | 42 | 39.5 | 37 | 34.5 | 32 | 29.5 | 24.5 | 19.5 |
| 10 mM MgCl₂ | 0.25 | 0.25 | 0.25 | 0.25 | 0.25 | 0.25 | 0.25 | 0.25 | 0.25 | 0.25 |
| (μl) | | | | | | | | | | |
| 100 mM dNTPs | 2.5 | 5 | 7.5 | 10 | 12.5 | 15 | 17.5 | 20 | 25 | 30 |
| (μl) | | | | | | | | | | |

TABLE 16
Experimental set-up for pH measurements in 20 mM MgCl₂

| | [dNTP] (mM) | | | | | | | | | |
|---|---|---|---|---|---|---|---|---|---|---|
| components | 5 | 10 | 15 | 20 | 25 | 30 | 35 | 40 | 50 | 60 |
| 5 mM NaOH | 0.25 | 0.25 | 0.25 | 0.25 | 0.25 | 0.25 | 0.25 | 0.25 | 0.25 | 0.25 |
| (μl) | | | | | | | | | | |
| H₂O (μl) | 46.75 | 44.25 | 41.75 | 39.25 | 36.75 | 34.25 | 31.75 | 29.25 | 24.25 | 19.25 |
| 10 mM MgCl₂ | 0.5 | 0.5 | 0.5 | 0.5 | 0.5 | 0.5 | 0.5 | 0.5 | 0.5 | 0.5 |
| (μl) | | | | | | | | | | |
| 100 mM dNTPs | 2.5 | 5 | 7.5 | 10 | 12.5 | 15 | 17.5 | 20 | 25 | 30 |
| (μl) | | | | | | | | | | |

TABLE 17
Experimental set-up for pH measurements in 30 mM MgCl₂

| | [dNTP] (mM) | | | | | | | | | |
|---|---|---|---|---|---|---|---|---|---|---|
| components | 5 | 10 | 15 | 20 | 25 | 30 | 35 | 40 | 50 | 60 |
| 5 mM NaOH | 0.25 | 0.25 | 0.25 | 0.25 | 0.25 | 0.25 | 0.25 | 0.25 | 0.25 | 0.25 |
| (μl) | | | | | | | | | | |
| H₂O (μl) | 46.25 | 43.75 | 41.25 | 38.75 | 36.25 | 33.75 | 31.25 | 28.75 | 23.75 | 18.75 |
| 10 mM MgCl₂ | 1 | 1 | 1 | 1 | 1 | 1 | 1 | 1 | 1 | 1 |
| (μl) | | | | | | | | | | |
| 100 mM dNTPs | 2.5 | 5 | 7.5 | 10 | 12.5 | 15 | 17.5 | 20 | 25 | 30 |
| (μl) | | | | | | | | | | |

The reaction components were mixed in the proportions shown in the above tables to give a final volume of 50 μl. pH of the mixtures was then measured using a Mettler Toledo SevenCompact™ 5220 pH meter fitted with an InLab® Micro pH electrode.

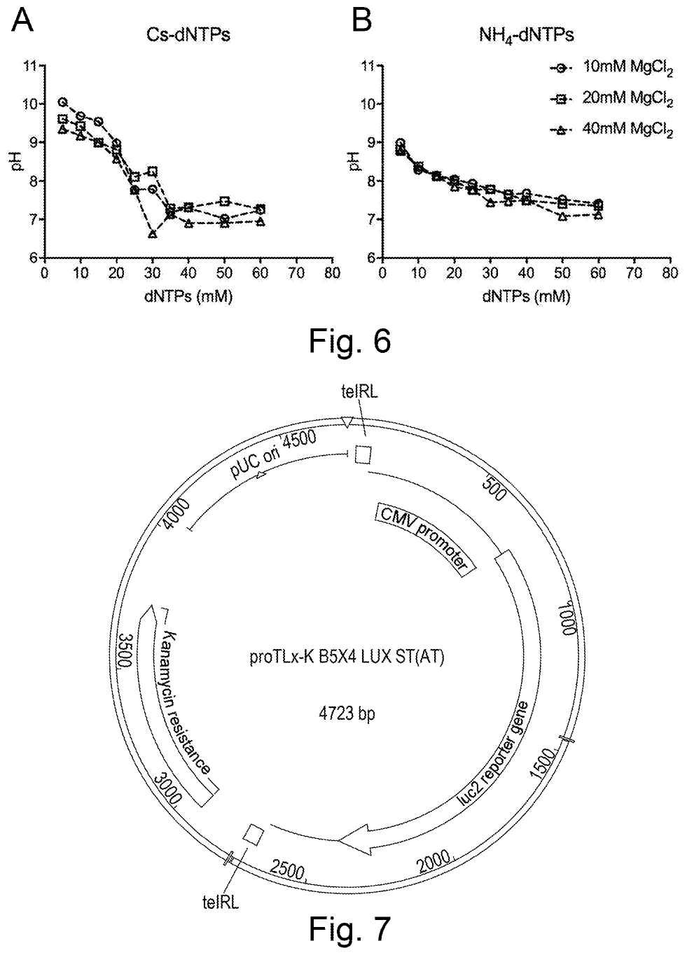

FIG. 6 shows the measured pHs of a range of dNTP concentrations (caesium and ammonium salts) in the presence of 10 mM, 20 mM and 40 mM MgCl₂. NaOH concentration is that used to denature the template DNA used in the DNA synthesis reaction. For the purposes of this experiment, all other DNA synthesis reaction components have been omitted since they are known not to influence starting pH. In all cases no specific pH stabilising buffer (e.g. Tris) has been added. The greater buffering capacity of the ammonium dNTPs over caesium dNTPs at dNTP salt concentrations of less than 30 mM is evident and expected. Interestingly, at concentrations of dNTP salts greater than 30 mM, the average pH for the caesium dNTP reactions and ammonium dNTP reactions are similar at about 7 and 7.5 respectively. The data suggests that the phosphate groups of the dNTPs themselves act to regulate the pH at about 7 when present at a sufficient concentration. Since the DNA polymerase enzyme can operate efficiently at about pH 7, this is an advantage for industrial scale synthesis reactions which require the use of high concentrations of dNTP salts. Importantly, it shows that for industrial scale reactions, DNA synthesis may be carried out with no or low concentrations of a specific buffer to achieve high productivities.

1. A cell-free process for the enzymatic synthesis of DNA comprising the use of nucleotide salts, wherein said salts comprise a monovalent cation with an ionic radius that is greater than that of the sodium ion.

2. A cell-free process according to claim 1 wherein said nucleotide salts are present at a concentration greater than 10 mM.

3. A cell-free process for the enzymatic synthesis of DNA comprising the use of nucleotide salts, wherein said nucleotide salts are present at a concentration of at least 10 mM and are either:
(a) a nucleotide salt comprising a monovalent cation whose ionic radius is greater than that of the sodium ion, or
(b) two or more nucleotide salts, each salt comprising different monovalent cations, wherein at least one of the cations has an ionic radius greater than that of the sodium ion.

4. A cell-free process according to any preceding claim wherein said nucleotide salts are present at a concentration of at least 15 mM, at least 20 mM, at least 25 mM, at least 30 mM, at least 35 mM or at least 40 mM.

5. A cell-free process according to any preceding claim wherein said monovalent cation or cations are independently selected from an alkali earth metal, a transition metal, or a polyatomic ion.

6. A cell-free process according to claim 5 wherein said monovalent cation or cations are independently selected from the list comprising potassium, ammonium, derivatives of ammonium, rubidium, caesium, or francium.

7. A cell-free process according to any preceding claim wherein said cell free process further comprises the use of one or more primers or a primase.

8. A cell-free process according to any preceding claim wherein said cell free process further comprises the use of a template.

9. A cell-free process for synthesising DNA according to any preceding claim wherein the cell-free process further comprises the use of one or more divalent metal cations, preferably selected from the list comprising magnesium, manganese, calcium, beryllium, zinc, and strontium.

10. A cell-free process according to claim 9 wherein the ratio between said divalent metal cations and the nucleotides is equal to or less than 1:1 in the reaction mixture, preferably less than 1:1.

11. A cell-free process according to any preceding claim wherein the process uses a maximum concentration of 10 mM sodium and/or lithium nucleotide salts.

12. A cell-free process according to any preceding claim wherein the process further comprises the use of a chemical denaturing agent, preferably sodium, potassium or ammonium hydroxide, and a pyrophosphatase.

13. A cell-free process according to claim 12 wherein no pH buffering agents are added to the process, and preferably no additional salts or detergents are added.

14. The cell-free process according to claim 13 wherein the nucleotide salts comprise caesium ions.

15. A cell-free process according to claim 12 wherein pH buffering agents, but no additional salts or detergents are added.

16. A cell-free process according to claim 15 wherein the nucleotide salts comprise ammonium ions.

17. The cell-free process according to any preceding claim wherein the process is for the synthesis of DNA on a large scale, preferably at least 3 g/l.

18. The use of nucleotide salts comprising caesium cations in the enzymatic cell-free synthesis of DNA.

19. The use of claim 18 wherein the enzymatic cell-free synthesis of DNA takes place in the presence of low levels of divalent cation, optionally at a ratio of 0.2:1 to 0.8:1 divalent cation to nucleotide, preferably 0.2:1 to 0.5:1.

20. The use of claim 18 wherein the cell-free synthesis of DNA takes place in minimal buffering agents, optionally comprising a pH buffer alone, without detergent or additional salts.

21. A method of amplifying a DNA template using a DNA polymerase wherein maintaining a ratio of divalent cations to nucleotides in the reaction mixture of 0.5:1 or below is required, comprising the use of nucleotide salts comprising caesium ions.

22. The use of nucleotide salts comprising rubidium cations in the enzymatic cell-free synthesis of DNA.

23. A cell-free method of amplifying a DNA template, said method comprising contacting said template and a DNA polymerase with nucleotides in the form of a salt in an amount equal to or greater than 40 mM, preferably greater than 60 mM or optionally greater than 80 mM wherein said salt comprises ammonium ions.

24. An enzymatic DNA synthesis which is performed under conditions of reduced concentrations of divalent cations, preferably magnesium, comprising the use of nucleotides in the form of salts comprising a monovalent cation with an ionic radius greater than that of the sodium ion.

25. A cell-free process, use, method, cell-free method or enzymatic synthesis of any preceding claim wherein the enzyme is a DNA polymerase, optionally a strand-displacement type polymerase.
